(19) United States
(12) Patent Application Publication
(10) Pub. No.: US 2015/0225711 A1
(43) Pub. Date: Aug. 13, 2015

(54) Factor VII Conjugates

(71) Applicant: NOVO NORDISK A/S, Bagsvaerd (DK)

(72) Inventors: Carsten Behrens, Koebenhavn (DK); Paul L. DeAngelis, Edmond, OK (US); Friedrich Michael Haller, Norman, OK (US)

(21) Appl. No.: 14/620,580

(22) Filed: Feb. 12, 2015

(30) Foreign Application Priority Data
Feb. 12, 2014 (EP) 14154875.0

(51) Int. Cl. C12N 9/64; A61K 47/48; A61K 38/48

(52) U.S. Cl. CPC C12N 9/6437; A61K 38/4846; A61K 47/4823

(57) ABSTRACT

The present invention relates to the conjugation of Factor VII polypeptides with heparosan polymers. The resultant conjugates may be used to deliver Factor VII, for example in the treatment or prevention of bleeding disorder

### FIELD OF THE INVENTION

The present invention relates to the conjugation of Factor VII polypeptides with heparosan polymers.

### CROSS-REFERENCE TO RELATED APPLICATIONS

This application claims priority under 35 U.S.C. §119 of European Patent Application 14154875.0, filed Feb. 12, 2014; the contents of which is incorporated herein by reference.

### SEQUENCE LISTING

The instant application contains a Sequence Listing which has been submitted in ASCII format via EFS-Web and is hereby incorporated by reference in its entirety. Said ASCII copy, created on Feb. 12, 2015, is named 130086US01_ST25.txt and is 4 kilobytes in size.

### SEQUENCE LISTING

SEQ ID NO: 1: Wild type human coagulation Factor VII.

### BACKGROUND TO THE INVENTION

An injury to a blood vessel activates the haemostatic system that involves complex interactions between cellular and molecular components. The process that eventually causes the bleeding to stop is known as haemostasis. An important part of haemostasis is coagulation of the blood and the formation of a clot at the site of the injury. The coagulation process is highly dependent on the function of several protein molecules. These are known as coagulation factors. Some of the coagulation factors are proteases which can exist in an inactive zymogen or an enzymatically active form. The zymogen form can be converted to its enzymatically active form by specific cleavage of the polypeptide chain catalyzed by another proteolytically active coagulation factor. Factor VII is a vitamin K-dependent plasma protein synthesized in the liver and secreted into the blood as a single-chain glycoprotein. The Factor VII zymogen is converted into an activated form (Factor VIIa) by specific proteolytic cleavage at a single site, i.e. between R152 and 1153 of the Factor VII sequence (wild type human coagulation Factor VII) resulting in a two chain molecule linked by a single disulfide bond. The two polypeptide chains in Factor VIIa are referred to as light and heavy chain, corresponding to residues 1-152 and 153-406, respectively, of the Factor VII sequence. Factor VII circulates predominantly as zymogen, but a minor fraction is on the activated form (Factor VIIa).

The blood coagulation process can be divided into three phases: initiation, amplification and propagation. The initiation and propagation phases contribute to the formation of thrombin, a coagulation factor with many important functions in haemostasis. The coagulation cascade starts if the single-layered barrier of endothelial cells that line the inner surface of blood vessels becomes damaged. This exposes subendothelial cells and extravascular matrix proteins to which platelets in the blood will stick to. If this happens, Tissue Factor (TF) which is present on the surface of sub-endothelial cells becomes exposed to Factor VIIa circulating in the blood. TF is a membrane-bound protein and serves as the receptor for Factor VIIa. Factor VIIa is an enzyme, a serine protease, with intrinsically low activity. However, when Factor VIIa is bound to TF, its activity increases greatly. Factor VIIa interaction with TF also localizes Factor VIIa on the phospholipid surface of the TF bearing cell and positions it optimally for activation of Factor X to Xa. When this happens, Factor Xa can combine with Factor Va to form the so-called “prothombinase” complex on the surface of the TF bearing cell. The prothrombinase complex then generates thrombin by cleavage of prothrombin.

The pathway activated by exposing TF to circulating Factor VIIa and leading to the initial generation of thrombin is known as the TF pathway. The TF:Factor VIIa complex also catalyzes the activation of Factor IX to Factor IXa. Then activated Factor IXa can diffuse to the surface of platelets which are sticking to the site of the injury and have been activated. This allows Factor IXa to combine with FVIIIa to form the “tenase” complex on the surface of the activated platelet. This complex plays a key role in the propagation phase due to its remarkable efficiency in activating Factor X to Xa. The efficient tenase catalyzed generation of Factor Xa activity in turn leads to efficient cleavage of prothrombin to thrombin catalyzed by the prothrombinase complex.

If there are any deficiencies in either Factor IX or Factor VIII, it compromises the important tenase activity, and reduces the production of the thrombin which is necessary for coagulation. Thrombin formed initially by the TF pathway serves as a pro-coagulant signal that encourages recruitment, activation and aggregation of platelets at the injury site. This results in the formation of a loose primary plug of platelets. However, this primary plug of platelets is unstable and needs reinforcement to sustain haemostasis. Stabilization of the plug involves anchoring and entangling the platelets in a web of fibrin fibres.

The formation of a strong and stable clot is dependent on the generation of a robust burst of local thrombin activity. Thus, deficiencies in the processes leading to thrombin generation following a vessel injury can lead to bleeding disorders e.g. haemophilia A and B. People with haemophilia A and B lack functional Factor VIIIa or Factor IXa, respectively. Thrombin generation in the propagation phase is critically dependent of tenase activity, i.e. requires both Factor VIIIa and FIXa. Therefore, in people with haemophilia A or B proper consolidation of the primary platelet plug fails and bleeding continues.

Replacement therapy is the traditional treatment for hemophilia A and B, and involves intravenous administration of Factor VIII or Factor IX. In many cases, however, patients develop antibodies (also known as inhibitors) against the infused proteins, which reduce or negate the efficacy of the treatment.

Recombinant Factor VIIa (Novoseven®) has been approved for the treatment of hemophilia A or B patients that have inhibitors, and also is used to stop bleeding episodes or prevent bleeding associated with trauma and/or surgery. Recombinant Factor VIIa also has been approved for the treatment of patients with congenital Factor VII deficiency.

According to the model that recombinant FVIIa operates through a TF-independent mechanism, recombinant FVIIa is directed to the surface of the activated blood platelets by virtue of its Gla-domain where it then proteolytically activates Factor X to Xa thus by-passing the need for a functional tenase complex. The low enzymatic activity of FVIIa in the absence of TF as well as the low affinity of the Gla-domain for membranes could explain the need for supra-physiological levels of circulating FVIIa needed to achieve haemostasis.

Recombinant Factor VIIa has a pharmacological half-life of 2-3 hours which may necessitate frequent administration to resolve bleedings in patients. Further, patients often only receive Factor VIIa therapy after a bleed has commenced, rather than as a precautionary measure, which often impinges upon their general quality of life. A recombinant Factor VIIa variant with a longer circulation half-life would decrease the number of necessary administrations and support less frequent dosing thus hold the promise of significantly improving Factor VIIa therapy to the benefit of patients and care-holders.

In general, there are many unmet medical needs in people with coagulopathies. The use of recombinant Factor VIIa to promote clot formation underlines its growing importance as a therapeutic agent. However, recombinant Factor VIIa therapy still leaves significant unmet medical needs, and there is a need for recombinant Factor VIIa polypeptides having improved pharmaceutical properties, for example increased in vivo functional half-life, improved activity, and less undesirable side effects.

Conjugation of half-life extending moieties—e.g. in the form of a hydrophilic polymer—with a peptide or polypeptide can be carried out by use of enzymatic methods. These methods can be selective, requiring the presence of specific peptide consensus motifs in the protein sequence, or the presence of post translational moieties such as glycans. Selective enzymatic methods for modifying N- and O-glycans on blood coagulation factors have been described. For example, chemically modified sialic acid substrates (Malmstrøm, J, Anal Bioanal Chem. 2012; 403:1167-1177) have been described that can be used to glycoPEGylate Factor VIIa on N-glycans using sialyltransferase ST3GalIII (Stennicke, H R. et al. Thromb Haemost. 2008 November; 100(5):920-8), and on O-glycans on Factor VIII using ST3GalI (Stennicke, H R. et al., Blood. 2013 Mar. 14; 121(11):2108-16). A common feature of the above mentioned methods is the use of a modified sialic acid substrate, glycyl sialic acid cytidine monophosphate (GSC), and the chemical acylation of GSC with the half-life extending moieties.

For example, PEG polymers activated as nitrophenyl- or N-hydroxy-succinimide esters can be acylated onto the glycyl amino group of GSC to create a PEG substituted sialic acid substrate that can be enzymatically transferred to the N- and O-glycans of glycoproteins (cf. WO2006127896, WO2007022512, US2006040856). In a similar way, fatty acids can be acylated onto the glycyl amino group of GSC using N-hydroxy-succinimide activated ester chemistry (WO2011101277).

However, the inventors have found that previously published methods are not suited for attaching highly functionalized half-life extending moieties such as carbohydrate polymers to GSC.

### SUMMARY OF THE INVENTION

Generally, the present invention derives from the finding that the polymer heparosan can be bound to Factor VII (FVII) in order to extend its half-life. An advantage with heparosan is that heparosan polymers are biodegradable, avoiding any potential accumulation problems related to non-biodegradable polymers. The use of heparosan polymers in this way can lead to improved properties of Factor VII polypeptide conjugates such as increased FIXa and FXa generation potential and improved clot activity.

Accordingly, the present invention provides a conjugate between a Factor VII polypeptide and a heparosan polymer.

In some embodiments, the polymer has a polydispersity index (Mw/Mn) of less than 1.10 or less than 1.05.

In another embodiment, the polymer has a size between 13 kDa and 65 kDa, such as 38 and 44 kDa.

The heparosan Factor VII polypeptide conjugate described herein may have increased circulating half-life compared to an un-conjugated Factor VII polypeptide; or increased functional half-life compared to an un-conjugated Factor VII polypeptide.

The heparosan Factor VII polypeptide conjugate described herein may have increased mean residence time compared to an un-conjugated Factor VII polypeptide; or increased functional mean residence time compared to an un-conjugated Factor VII polypeptide.

In some embodiments, the heparosan (HEP) Factor VII polypeptide conjugate described herein is produced using a linker which has improved properties (e.g., stability). In one such embodiment a HEP-FVII polypeptide conjugate is provided wherein the HEP moiety is linked to Factor VII in such a fashion that a stable and isomer free conjugate is obtained. In one such embodiment the HEP polymer is linked to Factor VII using a chemical linker comprising 4-methylbenzoyl connected to a sialic acid derivative such as glycyl sialic acid cytidine monophosphate (GSC).

The Factor VII polypeptide may be a variant of a Factor VII polypeptide carrying a free cysteine, such as FVIIa-407C, in which the heparosan polymer may be attached to the cysteine at position 407 of said Factor VII polypeptide. The polymer may be attached to the polypeptide via N- or O-glycans.

The Factor VII polypeptide may be a variant of a Factor VII polypeptide comprising two or more substitutions relative to the amino acid sequence of human Factor VII (SEQ ID NO:1), wherein T293 is replaced by Lys (K), Arg (R), Tyr (Y) or Phe (F) and L288 is replaced by Phe (F), Tyr (Y), Asn (N), or Ala (A) and/or W201 is replaced by Arg (R), Met (M) or Lys (K) and/or K337 is replaced by Ala (A) or Gly (G).

The Factor VII polypeptide may comprise a substitution of T293 with Lys (K) and a substitution of L288 with Phe (F). The Factor VII polypeptide may comprise a substitution of T293 with Lys (K) and a substitution of L288 with Tyr (Y). The Factor VII polypeptide may comprise a substitution of T293 with Arg (R) and a substitution of L288 with Phe (F). The Factor VII polypeptide may comprise a substitution of T293 with Arg (R) and a substitution of L288 with Tyr (Y). The Factor VII polypeptide may comprise, or may further comprise, a substitution of K337 with Ala (A). The Factor VII polypeptide may comprise a substitution of T293 with Lys (K) and a substitution of W201 with Arg (R).

The invention also provides compositions comprising the conjugates described herein, such as a pharmaceutical composition comprising a conjugate described herein and a pharmaceutically acceptable carrier or diluent.

A conjugate or composition described herein may be provided for use in a method of treating or preventing a bleeding disorder. That is, the invention relates to methods of treating or preventing a bleeding disorder, wherein said methods comprise administering a suitable dose of a conjugate described herein to a patient in need thereof, such as an individual in need of Factor VII, such as an individual having haemophilia A or haemophilia B.

### BRIEF DESCRIPTION OF THE FIGURES

FIG. 1a: Structure of heparosan.

FIG. 1b: Structure of a heparosan polymer with maleimide functionality at its reducing end.

FIG. 2a: Assessment of conjugate purity by SDS-PAGE. SDS-PAGE analysis of final FVIIa conjugates. Gel was loaded with HiMark HMW standard (lane 1); FVIIa (lane 2); 13k-HEP-[C]-FVIIa (lane 3); 27k-HEP-[C]-FVIIa (lane 4); 40k-HEP-[C]-FVIIa (lane 5); 52k-HEP-[C]-FVIIa (lane 6); 60k-HEP-[C]-FVIIa (lane 7); 65k-HEP-[C]-FVIIa (lane 8); 108k-HEP-[C]-FVIIa (lane 9) and 157k-HEP-[C]-FVIIa407C (lane 10).

FIG. 2b: Assessment of conjugate purity by SDS-PAGE. SDS-PAGE of glycoconjugated 52k-HEP-[N]-FVIIa. Gel was loaded with HiMark HMW standard (lane 1), ST3Gal3 (lane 2), FVIIa (lane 3), asialo FVIIa (lane 4), and 52k-HEP-[N]-FVIIa (lane 5).

FIG. 3: Analysis of FVIIa clotting activity levels of heparosan conjugates and glycoPEGylated FVIIa references.

FIG. 4: Proteolytic activity of heparosan conjugates and glycoPEGylated FVIIa references.

FIG. 5: PK results (LOCI) in Sprague Dawley rats. Comparison of unmodified FVIIa (2 studies), 13k-HEP-[C]-FVIIa407C, 27k-HEP-[C]-FVIIa407C, 40k-HEP-[C]-FVIIa407C, 52k-HEP-[C]-FVIIa407C, 65k-HEP-[C]-FVIIa407C, 108k-HEP-[C]-FVIIa407C and 157k-HEP-[C]-FVIIa407C, glycoconjugated 52k-HEP-[N]-FVIIa and reference molecules (40 kDa-PEG-[N]-FVIIa (2 studies) and 40 kDa-PEG-[C]-FVIIa407C). Data are shown as mean±SD (n=3-6) in a semilogarithmic plot.

FIG. 6: PK results (Clot Activity) in Sprague Dawley rats. Comparison of unmodified FVIIa (2 studies), 13k-HEP-[C]-FVIIa407C, 27k-HEP-[C]-FVIIa407C, 40k-HEP-[C]-FVIIa407C, 52k-HEP-[C]-FVIIa407C, 65k-HEP-[C]-FVIIa407C, 108k-HEP-[C]-FVIIa407C and 157k-HEP-[C]-FVIIa407C, glycoconjugated 52k-HEP-[N]-FVIIa and reference molecules (40 kDa-PEG-[N]-FVIIa (2 studies) and 40 kDa-PEG-[C]-FVIIa407C). Data are shown in a semilogarithmic plot.

FIG. 7: Relationship between HEP-size and mean residence time (MRT) for a number of HEP-[C]-FVIIa407C conjugates. MRT values from PK studies are plotted against heparosan polymer size of conjugates. The plot represent values for non-conjugated FVIIa, 13k-HEP-[C]-FVIIa407C, 27k-HEP-[C]-FVIIa407C, 40k-HEP-[C]-FVIIa407C, 52k-HEP-[C]-FVIIa407C, 65k-HEP-[C]-FVIIa407C, 108k-HEP-[C]-FVIIa407C and 157k-HEP-[C]-FVIIa407C. MRT (LOCI) was calculated by non-compartmental methods using Phoenix WinNonlin 6.0 (Pharsight Corporation).

FIG. 8: Functionalization of glycylsialic acid cytidine monophosphate (GSC) with a benzaldehyde group. GSC is acylated with 4-formylbenzoic acid and subsequently reacted with heparosan (HEP)-amine by a reductive amination reaction.

FIG. 9: Functionalization of heparosan (HEP) polymer with a benzaldehyde group and subsequent reaction with glycylsialic acid cytidine monophosphate (GSC) in a reductive amination reaction.

FIG. 10: Functionalization of glycylsialic acid cytidine monophosphate (GSC) with a thio group and subsequent reaction with a maleimide functionalized heparosan (HEP) polymer.

FIG. 11: Heparosan (HEP)—glycylsialic acid cytidine monophosphate (GSC).

FIG. 12: PK results (LOCI) in Sprague Dawley rats. Comparison of glycoconjugated 2×20k-HEP-[N]-FVIIa, 1×40k-HEP-[N]-FVIIa and reference molecule 1×40k-PEG-[N]-FVIIa. Data are shown as mean±SD (n=3-6) in a semilogarithmic plot.

FIG. 13: PK results (Clot Activity) in Sprague Dawley rats. Comparison of glycoconjugated 2×20k-HEP-[N]-FVIIa, 1×40k-HEP-[N]-FVIIa and reference molecule 1×40k-PEG-[N]-FVIIa. Data are shown as mean±SD (n=3-6) in a semilogarithmic plot.

FIG. 14: Reaction scheme wherein an asialoFactor VII glycoprotein is reacted with HEP-GSC in the presence of a ST3GalIII sialyltransferase.

### DETAILED DESCRIPTION OF THE INVENTION

The invention relates to conjugates between Factor VII (FVII) polypeptides and heparosan (HEP) polymers, as well as to methods for preparing such conjugates and uses for such conjugates. The Inventors have surprisingly found that Factor VII-heparosan conjugates have improved properties.

### Factor VII Polypeptides

The terms “Factor VII” or “FVII” denote Factor VII polypeptides. Suitable polypeptides may be produced by methods including natural source extraction and purification, and by recombinant cell culture systems. The sequence and characteristics of wild-type human Factor VII are set forth, for example, in U.S. Pat. No. 4,784,950.

Also encompassed within the term “Factor VII polypeptide” are biologically active factor VII equivalents and modified forms of Factor VII, e.g., differing in one or more amino acid(s) in the overall sequence. Furthermore, the terms used in this application are intended to cover substitution, deletion and insertion amino acid variants of Factor VII or posttranslational modifications.

As used herein, “Factor VII polypeptide” encompasses, without limitation, Factor VII, as well as Factor VII-related polypeptides. Factor VII-related polypeptides include, without limitation, Factor VII polypeptides that have either been chemically modified relative to human Factor VII and/or contain one or more amino acid sequence alterations relative to human Factor VII (i.e., Factor VII variants), and/or contain truncated amino acid sequences relative to human Factor VII (i.e., Factor VII fragments). Such factor VII-related polypeptides may exhibit different properties relative to human Factor VII, including stability, phospholipid binding, altered specific activity, and the like.

The term “Factor VII” is intended to encompass Factor VII polypeptides in their uncleaned (zymogen) form, as well as those that have been proteolytically processed to yield their respective bioactive forms, which may be designated Factor VIIa. Typically, Factor VII is cleaved between residues 152 and 153 to yield Factor VIIa.

The term “Factor VII” is also intended to encompass, without limitation, polypeptides having the amino acid sequence 1-406 of wild-type human Factor VII (as disclosed in U.S. Pat. No. 4,784,950), as well as wild-type Factor VII derived from other species, such as, e.g., bovine, porcine, canine, murine, and salmon Factor VII. It further encompasses natural allelic variations of Factor VII that may exist and occur from one individual to another. Also, degree and location of glycosylation or other post-translation modifications may vary depending on the chosen host cells and the nature of the host cellular environment.

As used herein, “Factor VII-related polypeptides” encompasses, without limitation, polypeptides exhibiting substantially the same or improved biological activity relative to wild-type human Factor VII. These polypeptides include, without limitation, Factor VII or Factor VIIa that has been chemically modified and Factor VII variants into which specific amino acid sequence alterations have been introduced that modify or disrupt the bioactivity of the polypeptide.

Also encompassed are polypeptides with a modified amino acid sequence, for instance, polypeptides having a modified N-terminal end including N-terminal amino acid deletions or additions, and/or polypeptides that have been chemically modified relative to human Factor VIIa.

Also encompassed are polypeptanides with a modified amino acid sequence, for instance, polypeptides having a modified C-terminal end including C-terminal amino acid deletions or additions, and/or polypeptides that have been chemically modified relative to human Factor VIIa.

Factor VII-related polypeptides, including variants of Factor VII, exhibiting substantially the same or better bioactivity than wild-type Factor VII, include, without limitation, polypeptides having an amino acid sequence that differs from the sequence of wild-type Factor VII by insertion, deletion, or substitution of one or more amino acids.

Factor VII-related polypeptides, including variants, having substantially the same or improved biological activity relative to wild-type Factor VIIa encompass those that exhibit at least about 25%, preferably at least about 50%, more preferably at least about 75%, more preferably at least about 100%, more preferably at least about 110%, more preferably at least about 120%, and most preferably at least about 130% of the specific activity of wild-type Factor VIIa that has been produced in the same cell type, when tested in one or more of a clotting assay, proteolysis assay, or TF binding assay.

The Factor VII polypeptide may be a Factor VII-related polypeptide, in particular a variant, wherein the ratio between the activity of said Factor VII polypeptide and the activity of native human Factor VIIa (wild-type FVIIa) is at least about 1.25 when tested in an in vitro hydrolysis assay; in other embodiments, the ratio is at least about 2.0; in further embodiments, the ratio is at least about 4.0. The Factor VII polypeptide may be a Factor VII analogue, in particular a variant, wherein the ratio between the activity of said Factor VII polypeptide and the activity of native human Factor VIIa (wild-type FVIIa) is at least about 1.25 when tested in an in vitro proteolysis assay; the ratio may be at least about 2.0; the ratio may be at least about 4.0; the ratio may be at least about 8.0.

The Factor VII polypeptide may be human Factor VII, as disclosed, e.g., in U.S. Pat. No. 4,784,950 (wild-type Factor VII). The Factor VII polypeptide may be human Factor VIIa. Factor VII polypeptides include polypeptides that exhibit at least about 90%, preferably at least about 100%, preferably at least about 120%, more preferably at least about 140%, and most preferably at least about 160%, of the specific biological activity of human Factor VIIa.

The Factor VII polypeptide may be a variant Factor VII polypeptide having a reduced interaction with antithrombin III when compared to that of human Factor VIIa. For example, the Factor VII polypeptide may have less than 100%, less than 95%, less than 90%, less than 80%, less than 70% or less than 50% of the interaction with antithrombin III of wild type human Factor VIIa. A reduced interaction with antithrombin III may be present in combination with another improved biological activity as described herein, such as an improved proteolytic activity.

The Factor VII polypeptide may have an amino acid sequence that differs from the sequence of wild-type Factor VII by insertion, deletion, or substitution of one or more amino acids.

The Factor VII polypeptide may be a polypeptide that exhibits at least about 70%, preferably at least about 80%, more preferably at least about 90%, and most preferable at least about 95%, of amino acid sequence identity with the sequence of wild-type Factor VII as disclosed in U.S. Pat. No. 4,784,950 (SEQ ID NO. 1: Wild type human coagulation Factor VII) Amino acid sequence homology/identity is conveniently determined from aligned sequences, using a suitable computer program for sequence alignment, such as, e.g., the ClustalW program, version 1.8, 1999 (Thompson et al., 1994, Nucleic Acid Research, 22: 4673-4680).

Non-limiting examples of Factor VII variants having substantially the same or improved biological activity as wild-type Factor VII include S52A-FVII, S60A-FVII (lino et al., Arch. Biochem. Biophys. 352: 182-192, 1998); L305V-FVII, L305V/M306D/D309S-FVII, L3051-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII; FVIIa variants exhibiting increased TF-independent activity as disclosed in WO 01/83725 and WO 02/22776; FVIIa variants exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999); and FVII variant polypeptides as disclosed in the PCT application EP2014/072076, for example FVII a variant polypeptide wherein the polypeptide comprise the following substitutions: L288F/T293K, L288F/T293K/K337A, L288F/T293R, L288F/T293R/K337A, L288Y/T293K, L288Y/T293K/K337A, L288Y/T293R, L288Y/T293R/K337A, L288N/T293K, L288N/T293K/K337A, L288N/T293R, L288N/T293R/K337A, W201R/T293K, W201R/T293K/K337A, W201R/T293R, W201R/T293R/K337A, W201R/T293Y, W201R/T293F, W201K/T293K or W201M/T293K.

Further Factor VII variants falling within the scope of Factor VII polypeptides herein are those described in WO 2007/031559 and WO 2009/126307.

Preferred Factor VII polypeptides for use in accordance with the present invention are those in which an additional cysteine residue has been added compared to an existing FVII sequence, such as a wild type FVII sequence. The cysteine may be appended to a Factor VII polypeptide at the C-terminal. The cysteine may be appended to a Factor VIIa polypeptide at the C-terminal residue 406 of the amino acid sequence of wild-type human Factor VII, leading to FVIIa 407C. The cysteine may be positioned in the amino acid sequence of a Factor VII molecule at a surface exposed position that will not seriously impede tissue factor binding, Factor X binding or binding to phospholipids. The structure of Factor VIIa is known and a suitable position meeting these requirements may therefore be identified by the skilled person.

The numbering of amino acids in the Factor VII polypeptide set out herein is based on the amino acid sequence for wild type human Factor VII as disclosed in U.S. Pat. No. 4,784,950 (SEQ ID NO. 1: Wild type human coagulation Factor VII). It will be apparent that equivalent positions in other Factor VII polypeptides may be readily identified by the skilled person by carrying out an alignment of the relevant sequences.

The biological activity of Factor VIIa in blood clotting derives from its ability to (i) bind to tissue factor (TF) and (ii) catalyze the proteolytic cleavage of Factor IX or Factor X to produce activated Factor IX or X (Factor IXa or Xa, respectively).

The biological activity of a Factor VII polypeptide may be measured by a number of ways as described below:

### Peptidolytic Activity Using Chromogenic Substrate (S-2288)

The peptidolytic activity of a FVII polypeptide or a FVII conjugate can be estimated using a chromogenic peptide (S-2288; Chromogenix) as substrate. A way of performing the assay is as follows: FVII polypeptide and appropriate FVIIa reference proteins are diluted in 50 mM HEPES, 5 mM CaCl₂, 100 mM NaCl, 0.01% Tween80, pH 7.4. The kinetic parameters for cleavage of the chromogenic substrate S-2288 are then determined in 96-well plate (n=3). In a typical experiment, 135 ul HEPES buffer, 10 μl of 200 nM FVIIa test entity solutions and 50 μl of 200 nM tissue factor stock solutions is added to the well. The micro plate is left for 5 minutes. The reaction is then initiated by addition of 10 μl of 10 mM S-2288 stock solution. The absorbance increase is measured continuously at 405 nm in a SpectraMax 190 microplate reader for 15 min. at room temperature. The amount of substrate converted is determined on the basis of a pNA (para-nitroaniline) standard curve. Relative activities are calculated from the initial rates, and compared to FVIIa rates. Activities for FVIIa conjugates can then be reported as a percentage of the activity of FVIIa reference.

### Proteolytic Activity Using Plasma-Derived Factor X as Substrate

The proteolytic activity of a FVII polypeptide or a FVII conjugate can be estimated using plasma-derived factor X (FX) as substrate. A way of performing the assay is as follows: All proteins are initially diluted in 50 mM HEPES (pH 7.4), 100 mM NaCl, 10 mM CaCl₂, 1 mg/mL BSA, and 0.1% (w/v) PEG8000. The kinetic parameters for FX activation are then determined by incubating 10 nM of each FVII polypeptide or conjugate with 40 nM FX in the presence of 25 uM PC:PS phospholipids (Haematologic technologies) for 30 min at room temperature in a total reaction volume of 100 μL in a 96-well plate (n=2). FX activation in the presence of soluble tissue factor (sTF) is determined by incubating 5 pM of each FVII polypeptide or FVII conjugate with 30 nM FX in the presence of 25 μM PC:PS phospholipids for 20 min at room temperature in a total reaction volume of 100 μL (n=2). After incubation, reactions are quenched by adding 50 μL stop buffer [50 mM HEPES (pH 7.4), 100 mM NaCl, 80 mM EDTA] followed by the addition of 50 μL 2 mM chromogenic peptide S-2765 (Chromogenix). Finally, the absorbance increase is measured continuously at 405 nm in a Spectramax 190 microplate reader. Catalytic efficiencies (k_{cat}/Kₘ) is determined by fitting the data to a revised form of the Michaelis Menten equation ([S]<Kₘ) using linear regression. The amount of FXa generated is estimated from a FXa standard curve.

### Assay for Measuring Clotting Time:

For the purposes of the invention, biological activity of Factor VII polypeptides (“Factor VII biological activity”) or of conjugates of the invention may also be quantified by measuring the ability of a preparation to promote blood clotting using Factor VII-deficient plasma and thromboplastin, as described, e.g., in U.S. Pat. No. 5,997,864 or WO 92/15686. In this assay, biological activity is expressed as the reduction in clotting time relative to a control sample and is converted to “Factor VII units” by comparison with a pooled human serum standard containing 1 unit/ml Factor VII activity.

### Assay for Determining Binding to Tissue Factor:

Alternatively, Factor VIIa biological activity may be quantified by measuring the physical binding of Factor VIIa or a Factor VII-related polypeptide to TF using an instrument based on surface plasmon resonance (Persson, FEBS Letts. 413:359-363, 1997).

### Potency as Measured by Soluble TF Dependent Plasma-Based FVIIa Clot Assay

Potencies can be estimated using a commercial FVIIa specific clotting assay; STACLOT®VIIa-rTF from Diagnostica Stago. The assay is based on the method published by J. H. Morrissey et al, Blood. 81:734-744 (1993). It measures sTF initiated FVIIa activity-dependent time to fibrin clot formation in FVII deficient plasma in the presence of phospholipids. Test compounds are diluted in Pipes+1% BSA assay dilution buffer and tested in 4 dilutions in 4 separate assay runs. Clotting times can be measured on an ACL9000 (ILS) coagulation instrument and results calculated using linear regression on a bilogarithmic scale based on a FVIIa calibration curve.

### Pharmacokinetic Evaluation in Sprauge Dawley Rats

The pharmacokinetic properties of a FVII polypeptide or a FVII conjugate can be estimated in sprauge Dawley rats. One way of performing such an animal study is as follows: The FVII polypeptide or FVII conjugate is initially formulated in a suitable buffer such as 10 mM Histidine, 100 mM NaCl, 10 mM CaCl₂, 0.01% Tween80 80, pH 6.0 and FVII polypeptide or FVII conjugate concentration in formulation buffer is determined by light chain quantification on HPLC. Male Sprague Dawley rats are obtained for the study. The animals are allowed at least one week acclimatisation period, and are allowed free access to feed and water before start of the experiment. The FVII polypeptide or FVII conjugate formulations are then given as a single iv bolus injection in the tail vein. Blood is then samples according to a predetermined schedual. Blood can be sampled the following way: 45 μl of blood is transferred to an Eppendorf tube containing 5 μl Stabilyte; 200 μl PIPES buffer (0.050 M Pipes, 0.10 M sodium chloride, 0.002 M EDTA, 1% (w/v) BSA, pH 7.2.) is added and inverted gently 5 times. The diluted citrate-stabilised blood is kept at room temperature until centrifugation at 4000 G for 10 minutes at room temperature. After centrifugation the supernatant is divided to three Micronic tubes; 70 ul for clot activity, 70 ul for antigen analysis and the rest as extra sample. The samples are immediately frozen on dry ice and storage at −80° C. until plasma analysis for example as described below can be carried out.

### Plasma Analysis; FVIIa-Clot Activity Level

FVIIa clotting activity levels of FVII polypeptide or a FVII conjugate in rat plasma can be estimated using a commercial FVIIa specific clotting assay; such as STACLOT®VIIa-rTF from Diagnostica Stago. The assay is based on the method published by J. H. Morrissey et al, Blood. 81:734-744 (1993). It measures soluble tissue factor (sTF) initiated FVIIa activity-dependent time to fibrin clot formation in FVII deficient plasma in the presence of phospholipids. Samples can be measured on an ACL9000 coagulation instrument against FVIIa calibration curves with the same matrix as the diluted samples (like versus like).

### Plasma Analysis; Antigen Concentration

FVII polypeptide or FVII conjugate antigen concentrations in plasma can be determined using LOCI technology. In this method, two monoclonal antibodies against human FVII are used for detection. The principle is described in Thromb Haemost 100(5):920-8 (2008). Samples are measured against drug substance calibration curves.

### Pharmacokinetic Analysis

Pharmacokinetic analysis can be carried out by non-compartmental methods (NCA) using for example WinNonlin (Pharsight Corporation St. Louis, Mo.) software. From the data the following parameters can be estimated: Cₘₐₓ (maximum concentration), Tₘₐₓ (time of maximum concentration), AUC (area under the curve from zero to infinity), AUCₑₓₜᵣₐₚ (percentage of AUC that are extrapolated from the last concentration to infinity), T_{1/2} (half-life), Cl (clearance) Vz (volume of distribution), and MRT (mean residence time).

These methods set out a comparison between a Factor VII polypeptide and wild-type Factor VIIa. However, it will be apparent that the same methods can also be used to compare the activity of a Factor VII polypeptide of interest with any other Factor VII polypeptide. For example, such a method may be used to compare the activity of a conjugate as described herein with a suitable control molecule such as an unconjugated Factor VII polypeptide, a Factor VII polypeptide that is conjugated with a water soluble polymer other than heparosan or a Factor VII polypeptide that is conjugated to a PEG, such as a 40 kDa PEG, rather than conjugated to heparosan. A method described herein, such as an in vitro hydrolysis assay or an in vitro proteolysis assay can therefore be adapted by substituting the Factor VIIa wild type polypeptide in the above methods with the control molecule of interest.

The ability of factor VIIa or Factor VII polypeptides to generate thrombin can also be measured in an assay comprising all relevant coagulation factors and inhibitors at physiological concentrations (minus factor VIII when mimicking hemophilia A conditions) and activated platelets (as described on p. 543 in Monroe et al. (1997) Brit. J. Haematol. 99, 542-547, which is hereby incorporated as reference)

The activity of the Factor VII polypeptides may also be measured using a one-stage clot assay (assay 4) essentially as described in WO 92/15686 or U.S. Pat. No. 5,997,864. Briefly, the sample to be tested is diluted in 50 mM Tris (pH 7.5), 0.1% BSA and 100 μl is incubated with 100 μl of Factor VII deficient plasma and 200 μl of thromboplastin C containing 10 mM Ca²⁺. Clotting times are measured and compared to a standard curve using a reference standard or a pool of citrated normal human plasma in serial dilution.

Human purified Factor VIIa suitable for use in the present invention may be made by DNA recombinant technology, e.g. as described by Hagen et al., Proc. Natl. Acad. Sci. USA 83: 2412-2416, 1986, or as described in European Patent No. 200.421 (ZymoGenetics, Inc.). Factor VII may also be produced by the methods described by Broze and Majerus, J. Biol. Chem. 255 (4): 1242-1247, 1980 and Hedner and Kisiel, J. Clin. Invest. 71: 1836-1841, 1983. These methods yield Factor VII without detectable amounts of other blood coagulation factors. An even further purified Factor VII preparation may be obtained by including an additional gel filtration as the final purification step. Factor VII is then converted into activated factor VIIa by known means, e.g. by several different plasma proteins, such as factor XIIa, IX a or Xa Alternatively, as described by Bjoern et al. (Research Disclosure, 269 September 1986, pp. 564-565), factor VII may be activated by passing it through an ion-exchange chromatography column, such as Mono Q(R) (Pharmacia fine Chemicals) or the like, or by autoactivation in solution.

Factor VII-related polypeptides may be produced by modification of wild-type Factor VII or by recombinant technology. Factor VII-related polypeptides with altered amino acid sequence when compared to wild-type Factor VII may be produced by modifying the nucleic acid sequence encoding wild-type factor VII either by altering the amino acid codons or by removal of some of the amino acid codons in the nucleic acid encoding the natural factor VII by known means, e.g. by site-specific mutagenesis.

The introduction of a mutation into the nucleic acid sequence to exchange one nucleotide for another nucleotide may be accomplished by site-directed mutagenesis using any of the methods known in the art. Particularly useful is the procedure that utilizes a super coiled, double stranded DNA vector with an insert of interest and two synthetic primers containing the desired mutation. The oligonucleotide primers, each complementary to opposite strands of the vector, extend during temperature cycling by means of Pfu DNA polymerase. On incorporation of the primers, a mutated plasmid containing staggered nicks is generated. Following temperature cycling, the product is treated with Dpnl, which is specific for methylated and hemimethylated DNA to digest the parental DNA template and to select for mutation-containing synthesized DNA. Other procedures known in the art for creating, identifying and isolating variants may also be used, such as, for example, gene shuffling or phage display techniques.

Separation of polypeptides from their cell of origin may be achieved by any method known in the art, including, without limitation, removal of cell culture medium containing the desired product from an adherent cell culture; centrifugation or filtration to remove non-adherent cells; and the like.

Optionally, Factor VII polypeptides may be further purified. Purification may be achieved using any method known in the art, including, without limitation, affinity chromatography, such as, e.g., on an anti-Factor VII antibody column (see, e.g., Wakabayashi et al., J. Biol. Chem. 261:11097, 1986; and Thim et al., Biochem. 27:7785, 1988); hydrophobic interaction chromatography; ion-exchange chromatography; size exclusion chromatography; electrophoretic procedures (e.g., preparative isoelectric focusing (IEF), differential solubility (e.g., ammonium sulfate precipitation), or extraction and the like. See, generally, Scopes, Protein Purification, Springer-Verlag, New York, 1982; and Protein Purification, J. C. Janson and Lars Ryden, editors, VCH Publishers, New York, 1989. Following purification, the preparation preferably contains less than about 10% by weight, more preferably less than about 5% and most preferably less than about 1%, of non-Factor VII polypeptides derived from the host cell.

Factor VII polypeptides may be activated by proteolytic cleavage, using Factor XIIa or other proteases having trypsin-like specificity, such as, e.g., Factor IXa, kallikrein, Factor Xa, and thrombin. See, e.g., Osterud et al., Biochem. 11:2853 (1972); Thomas, U.S. Pat. No. 4,456,591; and Hedner et al., J. Clin. Invest. 71:1836 (1983). Alternatively, Factor VII polypeptides may be activated by passing it through an ion-exchange chromatography column, such as Mono Q(R) (Pharmacia) or the like, or by autoactivation in solution. The resulting activated Factor VII polypeptide may then be conjugated with a heparosan polymer, formulated and administered as described in the present application.

### Heparosan Polymers

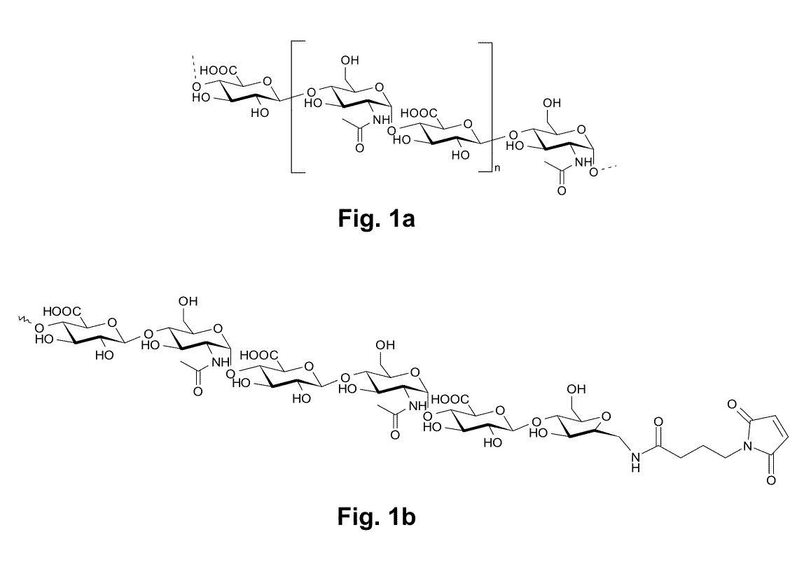

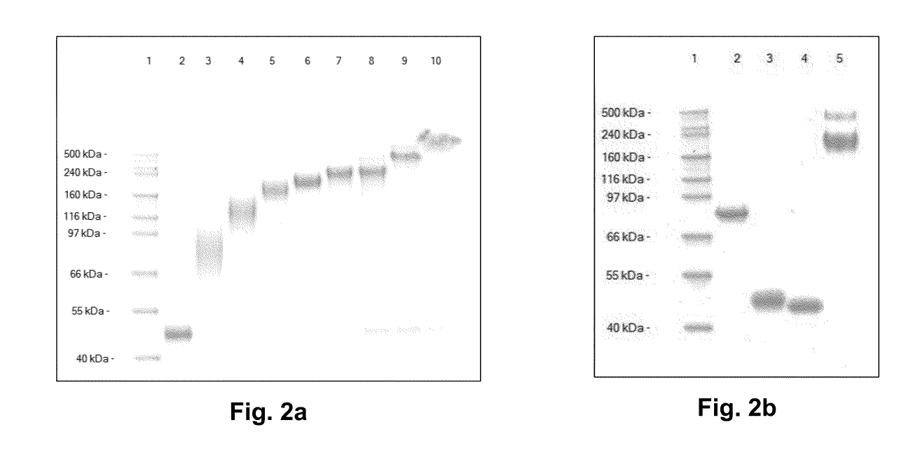

Heparosan (HEP) is a natural sugar polymer comprising (-GlcUA-beta1,4-GlcNAc-alpha1,4-) repeats (see FIG. 1A). HEP belongs to the glycosaminoglycan polysaccharide family and is a negatively charged polymer at physiological pH. HEP can be found in the capsule of certain bacteria but it is also found in higher vertebrate where it serves as precursor for the natural polymers heparin and heparan sulphate. HEP can be degraded by lysosomal enzymes such as N-acetyl-a-D-glucosaminidase (NAGLU) and β-glucuronidase (GUSB). An injection of a 100 kDa heparosan polymer labelled with Bolton-Hunter reagents has shown that heparosan is secreted as smaller fragments in body fluids/waste (US 2010/0036001).

Heparosan polymers and methods of making such polymers are described in US 2010/0036001, the content of which is incorporated herein by reference. In accordance with the present invention, the heparosan polymer may be any heparosan polymer described or disclosed in US 2010/0036001.

For use in the present invention, heparosan polymers can be produced by any suitable method, such as any of the methods described in US 2010/0036001 or US 2008/0109236. Heparosan can be produced using bacterial-derived enzymes. For example, the heparosan synthase PmHS 1 of Pasteurella mutocida Type D polymerises the heparosan sugar chain by transferring both GlcUA and GlcNAc. The Escherichia coli K5 enzymes KfiA (alpha GlcNAc transferase) and KfiC (beta GlcUA transferase) can together also form the disaccharide repeat of heparosan.

A heparosan polymer for use in the present invention is typically a polymer of the formula (-GlcUA-beta1,4-GlcNAc-alpha1,4-)ₙ. The size of the heparosan polymer may be defined by the number of repeats n in this formula. The number of said repeats n may be, for example, from 2 to about 5000. The number of repeats may be, for example 50 to 2000 units, 100 to 1000 units or 200 to 700 units. The number of repeats may be 200 to 250 units, 500 to 550 units or 350 to 400 units. Any of the lower limits of these ranges may be combined with any higher upper limit of these ranges to form a suitable range of numbers of units in the heparosan polymer.

The size of the heparosan polymer may be defined by its molecular weight. The molecular weight may be the average molecular weight for a population of heparosan polymer molecules, such as the weight average molecular mass.

Molecular weight values as described herein in relation to size of the heparosan polymer may not, in practise, exactly be the size listed. Due to batch to batch variation during heparosan polymer production, some variation is to be expected. To encompass batch to batch variation, it is therefore to be understood, that a variation around +/−10%, 9%, 8%, 7%, 6%, 5%, 4%, 3%, 2% or 1% around target HEP polymer size is to be expected. For example HEP polymer size of 40 kDa denotes 40 kDa+/−10%, e.g. 40 kDa could for example in practise mean 38.8 kDa or 41.5 kDa, both falling within the +/−10% range of 36 to 44 kDa of 40 kDa.

The heparosan polymer may have a molecular weight of, for example, 500 Da to 1,000 kDa. The molecular weight of the polymer may be 500 Da to 650 kDa, 5 kDa to 750 kDa, 10 kDa to 500 kDa, 15 kDa to 550 kDa or 25 kDa to 250 kDa.

The molecular weight may be selected at particular levels within these ranges in order to achieve a suitable balance between activity of the Factor VII polypeptide and half-life or mean residence time of the conjugate. For example, the molecular weight of the polymer may be in a range selected from 15-25 kDa, 25-35 kDa, 35-45 kDa, 45-55 kDa, 55-65 kDa or 65-75 kDa.

More specific ranges of molecular weight may be selected. For example, the molecular weight may be 20 kDa to 35 kDa, such as 22 kDa to 32 kDa such as 25 kDa to 30 kDa, such as about 27 kDa. The molecular weight may be 35 to 65 kDa, such as 40 kDa to 60 kDa, such as 47 kDa to 57 kDa, such as 50 kDa to 55 kDa such as about 52 kDa. The molecular weight may be 50 to 75 kDa such as 60 to 70 kDa, such as 63 to 67 kDa such as about 65 kDa.

In another embodiment, the heparosan polymer of the Factor VII conjugate, of the invention, has a size in a range selected from 13-65 kDa, 13-55 kDa, 25-55 kDa, 25-50 kDa, 25-45 kDa, 30-45 kDa and 38-42 kDa.

Any of the lower limits of these ranges of molecular weight may be combined with any higher upper limit from these ranges to form a suitable range for the molecular weight of the heparosan polymer as described herein.

The heparosan polymer may have a narrow size distribution (i.e. monodisperse) or a broad size distribution (i.e. polydisperse). The level of polydispersity (PDI) may be represented numerically based on the formula Mw/Mn, where Mw=weight average molecular mass and Mn=number average molecular weight. The polydispersity value using this equation for an ideal monodisperse polymer is 1. Preferably, a heparosan polymer for use in the present invention is monodisperse. The polymer may therefore have a polydispersity that is about 1, the polydispersity may be less than 1.25, preferably less than 1.20, preferably less than 1.15, preferably less than 1.10, preferably less than 1.09, preferably less than 1.08, preferably less than 1.07, preferably less than 1.06, preferably less than 1.05.

The molecular weight size distribution of the heparosan may be measured by comparison with monodisperse size standards (HA Lo-Ladder, Hyalose LLC) which may be run on agarose gels.

Alternatively, the size distribution of heparosan polymers may be determined by high performance size exclusion chromatography-multi angle laser light scattering (SEC-MALLS). Such a method can be used to assess the molecular weight and polydispersity of a heparosan polymer.

Polymer size may be regulated in enzymatic methods of production. By controlling the molar ratio of heparosan acceptor chains to UDP sugar, it is possible to select a final heparosan polymer size that is desired

The heparosan polymer may further comprise a reactive group to allow its attachment to a Factor VII polypeptide. A suitable reactive group may be, for example, an aldehyde, alkyne, ketone, maleimide, thiol, azide, amino, hydrazide, hydroxylamine, carbonate ester, chelator or a combination of any thereof. For example, FIG. 1B illustrates a heparosan polymer comprising a maleimide group.

Further examples of reactive groups that can be added to the heparosan polymer are as follows:
aldehyde reaction group added at the reducing terminus, reactive with amines
maleimide group added at the reducing terminus, reactive with sulfhydryls
pyridylthio group added at the reducing terminus, reactive with sulfhydryls
azido group added at the non-reducing terminus or within the sugar chain, reactive with acetylenes
amino group added at the reducing terminus, non-reducing terminus or within the sugar chain, reactive with aldehydes
N-hydroxy succinimide group added at the reducing or non-reducing terminus, reactive with amines

Hydroxylamine group added at the reducing or non-reducing terminus, react with aldehydes and ketones.
hydrazide added at the reducing terminus, reactive with aldehydres or ketones.

As set out in the Examples, maleimide functionalized heparosan polymers of defined size may be prepared by an enzymatic (PmHS1) polymerization reaction using the two sugar nucleotides UDP-GlcNAc and UDP-GlcUA in equimolar amount. A priming trisaccharide (GlcUA-GlcNAc-GlcUA)NH₂ may be used for initiating the reaction, and polymerization run until depletion of sugar nucleotide building blocks. Terminal amine (originating from the primer) may then be functionalized with suitable reactive groups such as a reactive group as described above, such as a maleimide functionality designed for conjugation to free cysteines. The size of the heparosan polymers can be pre-determined by variation in sugar nucleotide: primer stoichiometry. The technique is described in detail in US 2010/0036001.

The reactive group may be present at the reducing or non-reducing termini or throughout the sugar chain. The presence of only one such reactive group is preferred when conjugating the heparosan polymer to the polypeptide.

### Methods for Preparing FVII-HEP Conjugates

In some embodiments, a Factor VII polypeptide as described herein is conjugated to a heparosan polymer as described herein. Any Factor VII polypeptide as described herein may be combined with any heparosan polymer as described herein.

The heparosan polymer may be attached at a single position on the polypeptide, or heparosan polymers may be attached at multiple positions on the polypeptide.

The location of attachment of the polymer to the polypeptide may depend on the particular polypeptide molecule being used. The location of attachment of the polymer to the polypeptide may depend on the type of reactive group, if any, that is present on the polymer. As explained above, different reactive groups will react with different groups on the polypeptide molecule.

Various methods of attaching polymers to polypeptides exist and any suitable method may be used in accordance with the present invention. Heparosan polymers may be attached to the glycans of a Factor VII polypeptide using attachment technology described in any of US 2010/0036001, WO03/031464, WO2005/014035 or WO2008/025856, the content of each of which is included herein by reference.

For example, WO 03/031464 describes methods for remodelling the glycan structure of a polypeptide, such as a Factor VII or Factor VIIa polypeptide and methods for the addition of a modifying group such as a water soluble polymer to such a polypeptide. Such methods may be used to attach a heparosan polymer to a Factor VII polypeptide in accordance with the present invention.

As set out in the Examples, a Factor VII polypeptide may be conjugated to its glycan moieties using sialyltransferase. For enablement of this approach, a HEP polymer first need to be linked to a sialic acid cytidine monophosphate. Glycylsialic acid cytidine monophosphate (GSC) is a suitable starting point for such chemistry, but other sialic acid cytidine monophosphate or fragments of such can be used. Examples set out methods for covalent linking HEP polymers to GSC molecules. By covalent attachment, a HEP-GSC (HEP conjugated glycylsialic acid cytidine monophosphate) molecule is created that can be transferred to glycan moieties of FVIIa.

WO 2005/014035 describes chemical conjugation that utilises galactose oxidase in combination with terminal galactose-containing glycoproteins such as sialidase treated glycoproteins or asialo glycoproteins. Such method may utilise the reaction of sialidases and galactose oxidase to produce reactive aldehyde groups that can be chemically conjugated to nucleophilic reactive groups to attach a polymer to a glycoprotein. Such methods may be used to attach a heparosan polymer to a Factor VII glycoprotein. A suitable Factor VII polypeptide for use in such methods may be any Factor VII glycopeptide that comprises terminal galactose. Such a glycoprotein may be produced by treatment of a Factor VII polypeptide with sialidase to remove terminal sialic acid.

WO2011012850 describes the attachment of polymeric groups to a glycosyl group in a glycoprotein. Such methods may be used in accordance with the present invention to attach a heparosan polymer to a Factor VII polypeptide.

Heparosan may be attached to the polypeptide via an engineered extra cysteine in the polypeptide or an exposed sulfhydryl group. The sulfhydryl the cysteine group may be coupled to a functionalised heparosan polymer, such as a maleimide-heparosan polymer to obtain a heparosan-polypeptide conjugate.

In one aspect the heparosan polymer is attached to a FVII polypeptide by conjugation to a cysteine on the FVII molecule. The cysteine may be engineered into a Factor VII polypeptide, such as added to the amino acid sequence of a wild-type Factor VII polypeptide. The cysteine may be positioned at the C-terminal of the Factor VII polypeptide, such as at position 407, or in chain at a surface exposed position that will not seriously impede tissue factor binding, FX binding or binding to phospholipids.

In a Factor VII polypeptide that has been modified by addition of a cysteine residue at position 407, the Cys407 can act as site of attachment of a heparosan polymer (e.g. a 13 kDa, 27 kDa, 40 kDa, 52 kDa, 60 kDa, 65 kDa, 108 kDa or 157 kDa heparosan polymer that has been functionalised with maleimide).

As set out in the Examples, a Factor VII polypeptide with unblocked cysteine, such as FVIIa-407C, may be reacted with HEP-maleimide in a suitable buffer such as HEPES and at near neutral pH. The reaction may be allowed to stand at room temperature for, for example, 3-4 hours. Such a reaction can achieve the conjugation of the heparosan polymer to the Factor VII polypeptide.

Factor VII-heparosan conjugates may be purified once they have been produced. For example, purification may comprise by affinity chromatography using immobilised mAb directed towards the Factor VII polypeptide, such as mAb directed against the calcified gla-domain on FVIIa. In such an affinity chromatography method, unconjugated HEP-maleimide may be removed by extensive washing of the column. FVII may be released from the column by releasing the FVII from the antibody. For example, where the antibody is specific to the calcified gla-domain, release from the column may be achieved by washing with a buffer comprising EDTA.

Size exclusion chromatography may be used to separate Factor VII-heparosan conjugates from unconjugated Factor VII.

Pure conjugate may be concentrated by ultrafiltration.

Final concentrations of Factor VII-heparosan conjugate resulting from a process of production may be determined by, for example, HPLC quantification, such as HPLC quantification of the FVII light chain.

Common methods for linking half-life extending moieties such as carbohydrate polymers to glycoproteins comprise oxime, hydrazone or hydrazide bond formation. WO2006094810 describes methods for attaching hydroxyethyl starch polymers to glycoproteins such as erythropoietin that circumvent the problems connected to using activated ester chemistry. In these methods, hydroxyethyl starch and erythropoietin are individually oxidized with periodate on the carbohydrate moieties, and the reactive carbonyl groups ligated together using bis-hydroxylamine linking agents. The method will create hydroxyethyl starch linked to the erythropoietin via oxime bonds.

Similar oxime based linking methodology can be imagined for attaching carbohydrate polymers to GSC (WO2011101267), however, as such oxime bonds are known to exist in both syn- and anti-isomer forms, the linkage between the polymer and the protein will contain both syn- and anti-isomer combinations. Such isomer mixtures are usually not desirable in proteinaceous medicaments that are used for long term repeating administration since the linker inhomogeneity may pose a risk for antibody generation. Oxime and hydrazone bonds have also been shown to be instable in aquous solution (see for example Kalia and Raines, Angew Chem Int Ed Engl. 2008; 47(39): 7523-7526). The above mentioned methods have further disadvantages. In the oxidative process required for activating the glycoprotein, parts of the carbohydrate residues are chemically cleaved and the carbohydrates will therefore not be present in an intact form in the final conjugate. The oxidative process furthermore will generate product heterogenicity as the oxidating agent i.e. periodate in most cases is unspecific with regard to which glycan residue is oxidized. Both product heterogenicity and the presence of non-intact glycan residues in the final drug conjugate may impose immunogenicity risks.

Alternatives for linking carbohydrate polymers to glycoproteins involve the use of maleimide chemistry (WO2006094810). For example, the carbohydrate polymer can be furnished with a maleimido group, which selectively can react with a sulfhydryl group on the target protein. The linkage will then contain a cyclic succinimide group.

It is shown that it is possible to link a carbohydrate polymer such as HEP via a maleimido group to a thio-modified GSC molecule and transfer the reagent to an intact glycosyl groups on a glycoprotein by means of a sialyltransferase, thereby creating a linkage that contains a cyclic succinimide group.

Succinimide based linkages, however, may undergo hydrolytic ring opening when the conjugate is stored in aqueous solution for extended time periods (Bioconjugation Techniques, G. T. Hermanson, Academic Press, 3^{rd} edition 2013 p. 309) and while the linkage may remain intact, the ring opening reaction will add undesirable heterogeneity in form of regio- and stereo-isomers to the final conjugate.

It follows from the above that it is preferable to link the half-life extending moiety to the glycoprotein in such a way that 1) the glycan residue of the glycoprotein is preserved in intact form, and 2) no heterogenicity is present in the linker part between the intact glycosyl residue and the half-life extending moiety.

There is a need in the art for methods of conjugating two compounds, such as a half-life extending moiety such as HEP to a protein or protein glycan, wherein the compounds are linked such that a stable and isomer free conjugate is obtained.

In one aspect the present invention provides a stable and isomer free linker for use in sialic acid based conjugation of HEP to FVII wherein the HEP polymer may be attached to the sialic acid at positions appropriate for derivatization. Appropriate sites are known to the skilled person, or can be deduced from WO03031464 (which is hereby incorporated by reference in its entirety), wherein PEG polymers are attached to sialic acid cytidine monophosphate in multiple ways.

The HEP polymer may be attached to sialic acid at positions appropriate for derivatization. Appropriate sites are known to a skilled person, or can be deduced from WO03031464 (which is hereby incorporated by reference in its entirety), wherein polyethylene glycol polymers are attached to sialic acid cytidine monophosphate in multiple ways.

In some embodiments the C4 and C5 position of the sialic acid pyranose ring, as well as the C7, C8 and C9 position of the side chain can serve as points of derivatization. Derivatization preferably involves the existing hetero atoms of the sialic acid, such as the hydroxyl or amine group, but functional group conversion to render appropriate attachment points on the sialic acid is also a possibility.

In some embodiments, the 9-hydroxy group of the sialic acid N-acetylneuraminic acid may be converted to an amino group by methods known in the art. Eur. J. Biochem 168, 594-602 (1987). The resulting 9-deoxy-amino N-acetylneuraminic acid cytidine monophosphate as shown below is an activated sialic acid derivative that can serve as an alternative to GSC.

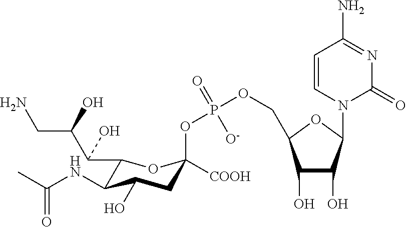

In some embodiments non-amine containing sialic acids such as 2-keto-3-deoxy-nonic acid, also known as KDN may also be converted to 9-amino derivatized sialic acids following same scheme.

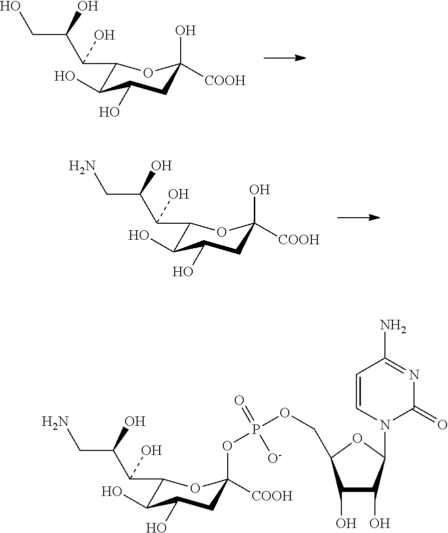

A similar scheme can be used for the shorter C8-sugar analogues belonging to the sialic acid family. Thus shorter versions of sialic acids such as 2-keto-3-deoxyoctonate, also known as KDO may be converted to the 8-deoxy-8-amino-2-keto-3-deoxyoctonate cytidine monophosphate, and used as an alternative to sialic acids that do not lack the C9 carbon atom.

In some embodiments, neuraminic acid cytidine monophosphate may be used in the invention. This material can be prepared as described in Eur. J. Org. Chem. 2000, 1467-1482.

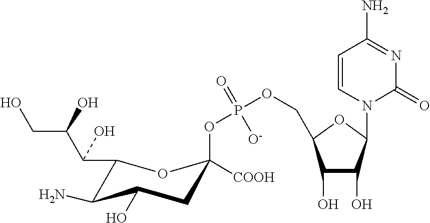

In some embodiments a stable and isomer free linker for use in glycyl sialic acid cytidine monophosphate (GSC) based conjugation of HEP to Factor VII is provided.

The GSC starting material used in the current invention can be synthesised chemically (Dufner, G. Eur. J Org Chem 2000, 1467-1482) or it can be obtained by chemoenzymatic routes as described in WO2007056191. The GSC structure is shown below:

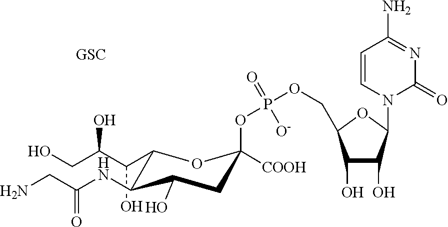

In some embodiments conjugates described herein comprise a linker comprising the following structure:

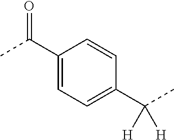

hereinafter also referred to as sublinker or sublinkage—that connects a HEP-amine and GSC in one of the following ways:

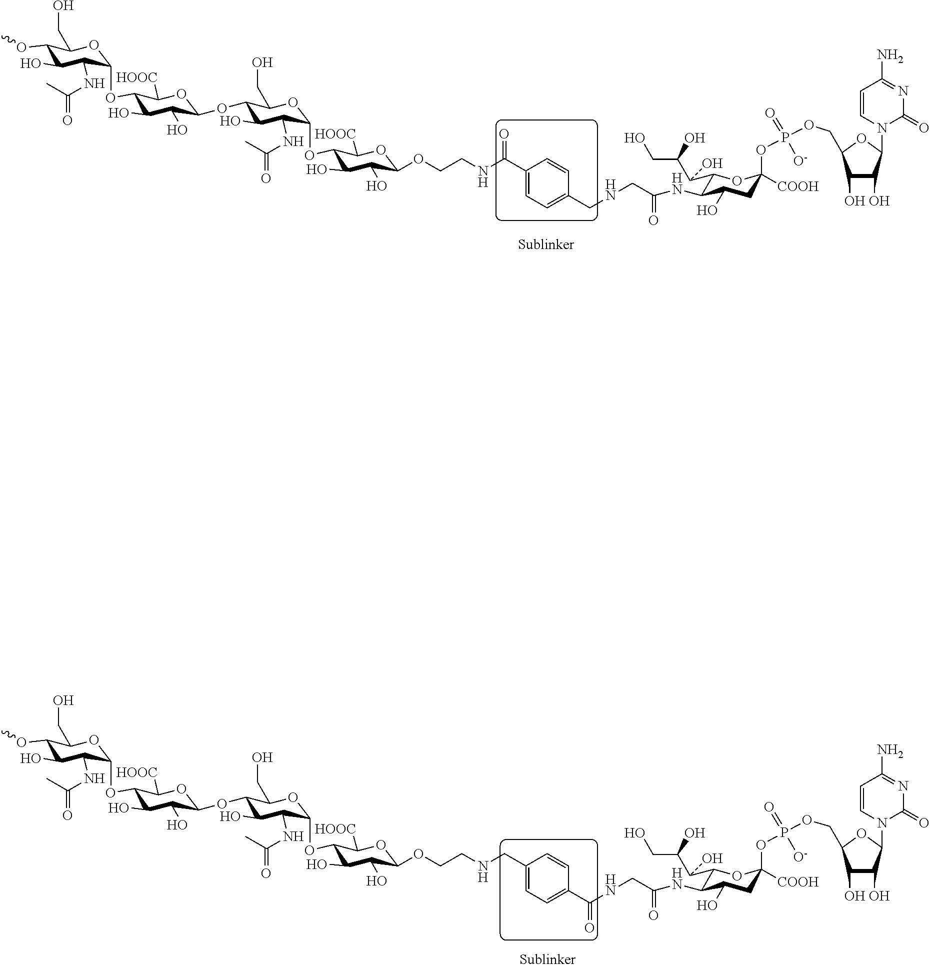

The highlighted 4-methylbenzoyl sublinker thus makes up part of the full linking structure linking the half-life extending moiety to a target protein. The sublinker is as such a stable structure compared to alternatives, such as succinimide based linkers (prepared from maleimide reactions with sulfhydryl groups) since the latter type of cyclic linkage has a tendency to undergo hydrolytic ring opening when the conjugate is stored in aqueous solution for extended time periods (Bioconjugation Techniques, G. T. Hermanson, Academic Press, 3^{rd} edition 2013 p. 309). Even though the linkage in this case (e.g. between HEP and sialic acid on a glycoprotein) may remain intact, the ring opening reaction will add heterogeneity in form of regio- and stereo-isomers to the final conjugate composition.

One advantage associated with conjugates described herein is thus that a homogenous composition is obtained, i.e. that the tendency of isomer formation due to linker structure and stability is significantly reduced. Another advantage is that the linker and conjugates according to the invention can be produced in a simple process, preferably a one-step process.

Isomers are undesirable since these can lead to a heterogeneous product and increase the risk for unwanted immune responses in humans.

The 4-methylbenzoyl sublinkage as used herein between HEP and GSC is not able to form sterio- or regio isomers. HEP polymers can be prepared by a synchronised enzymatic polymerisation reaction (US 20100036001). This method use heparan synthetase I from Pasturella multocida (PmHS1) which can be expressed in E. coli as a maltose binding protein fusion constructs. Purified MBP-PmHS1 is able to produce monodisperse polymers in a synchronized, stoichiometrically controlled reaction, when it is added to an equimolar mixture of sugar nucleotides (GlcNAc-UDP and GlcUA-UDP). A trisaccharide initiator (GlcUA-GlcNAc-GlcUA) is used to prime the reaction, and polymer length is determined by the primer:sugar nucleotide ratios. The polymerization reaction will run until about 90% of the sugar nucleotides are consumed. Polymers are isolated from the reaction mixture by anion exchange chromatography, and subsequently freeze-dried into stable powder.

Processes for preparation of functional HEP polymers are described in US 20100036001 which for example lists aldehyde-, amine- and maleimide functionalized HEP reagents. US 20100036001 is hereby incorporated by reference in its entirety as if fully set forth herein. A range of other functionally modified HEP derivatives are available using similar chemistry. HEP polymers used in certain embodiments of the present invention are initially produced with a primary amine handle at the reducing terminal according to methods described in US20100036001. HEP polymers with a primary amine handle (HEP-NH₂) can for example be prepared as described in Sismey-Ragatz et al., 2007 J Biol Chem and U.S. Pat. No. 8,088,604. Briefly, a fusion of the E. coli maltose-binding protein with PmHS1 is used as the catalyst to elongate heparosan oligosaccharide acceptors with a free amine at the reducing terminus into longer chains with UDP-GlcNAc and UDP-GlcUA precursors. The acceptor synchronizes the reaction so all chains are the same length (quasi-monodisperse size distribution) and it also imparts the free amine group to the sugar chain for subsequent modification or coupling reactions. Amine functionalized HEP polymers (i.e. HEP having an amine-handle) prepared according US20100036001 can be converted into a HEP-benzaldehyde by reaction with N-succinimidyl 4-formylbenzoate and subsequently coupled to the glycylamino group of GSC by a reductive amination reaction. The resulting HEP-GSC product can subsequently be enzymatically conjugated to a glycoprotein using a sialyltransferase.

For example said amine handle on HEP can be converted into a benzaldehyde functionality by reaction with N-succinimidyl 4-formylbenzoateaccording to the below scheme:

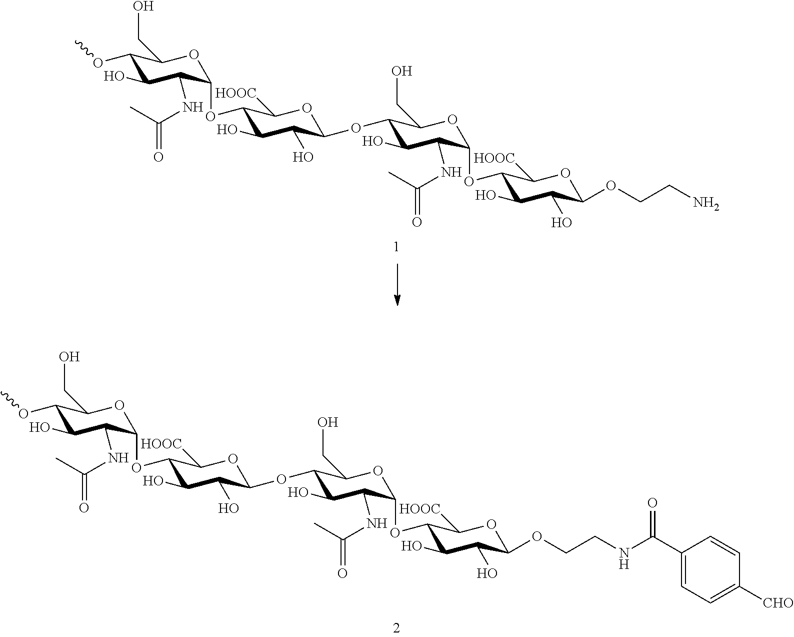

The conversion of HEP amine (1) to the 4-formylbenzamide compound (2) in the above scheme may be carried out by reaction with acyl activated forms of 4-formylbenzoic acid.

N-hydroxysuccinimidyl may be chosen as acyl activation group but a number of other acyl activation groups are known to the skilled person. Non-limited examples include 1-hydroxy-7-azabenzotriazole-, 1-hydroxy-benzotriazole-, pentafluorophenyl-esters as know from peptide chemistry.

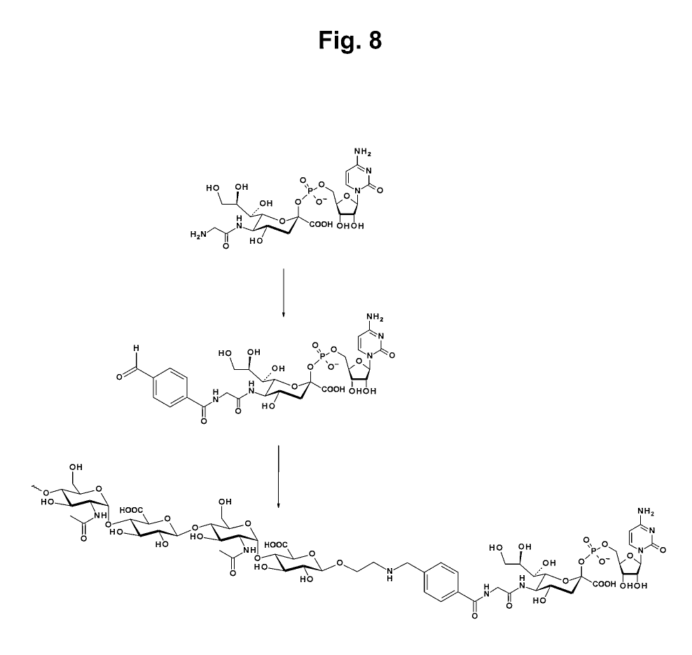

HEP reagents modified with a benzaldehyde functionality can be kept stable for extended time periods when stored frozen (−80° C.) in dry form. Alternatively, a benzaldehyde moiety can be attached to the GSC compound, thereby resulting in a GSC-benzaldehyde compound suitable for conjugation to an amine functionalized half-life extending moiety. This route of synthesis is depicted in FIG. 8.

For example, GSC can be reacted under pH neutral conditions with N-succinimidyl 4-formylbenzoate to provide a GSC compound that contains a reactive aldehyde group (see for example WO2011101267). The aldehyde derivatized GSC compound (GSC-benzaldehyde) can then be reacted with HEP-amine and reducing agent to form a HEP-GSC reagent.

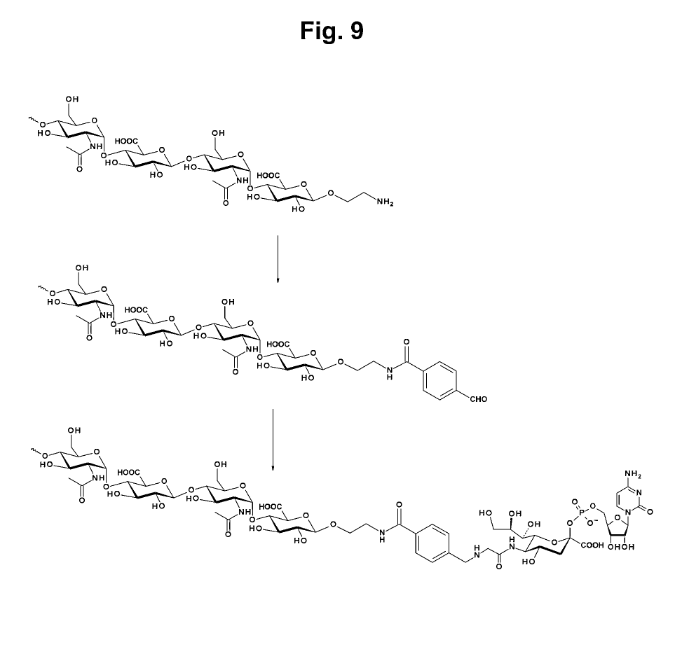

The above mentioned reaction may be reversed, so that the HEP-amine is first reacted with N-succinimidyl 4-formylbenzoate to form an aldehyde derivatized HEP-polymer, which subsequently is reacted directly with GSC in the presence of a reducing agent. In practice this eliminates the tedious chromatographic handling of GSC-CHO. This route of synthesis is depicted in FIG. 9.

Thus, in some embodiments HEP-benzaldehyde is coupled to GSC by reductive amination.

Reductive amination is a two-step reaction which proceeds as follows: Initially an imine (also known as Schiff-base) is formed between the aldehyde component and the amine component (in the present embodiment the glycyl amino group of GSC). The imine is then reduced to an amine in the second step. The reducing agent is chosen so that it selectively reduces the formed imine to an amine derivative.

A number of suitable reducing reagents are available to the skilled person. Non-limiting examples include sodium cyanoborohydride (NaBH3CN), sodium borohydride (NaBH4), pyridin boran complex (BH3:Py), dimethylsulfide boran complex (Me2S:BH3) and picoline boran complex.

Although reductive amination to the reducing end of carbohydrates (for example to the reducing termini of HEP polymers) is possible, it has generally been described as a slow and inefficient reaction (J C. Gildersleeve, Bioconjug Chem. 2008 July; 19(7): 1485-1490). Side reactions, such as the Amadori reaction, where the initially formed imine rearrange to a keto amine are also possible, and will lead to heterogenicity which as previously discussed is undesirable in the present context.

Aromatic aldehydes such as benzaldehydes derivatives are not able to form such rearrangement reactions as the imine is unable to enolize and also lack the required neighbouring hydroxy group typically found in carbohydrate derived imines Aromatic aldehydes such as benzaldehydes derivatives are therefore particular useful in reductive amination reactions for generating isomer free HEP-GSC reagent.

A surplus of GSC and reducing reagent is optionally used in order to drive reductive amination chemistry fast to completion. When the reaction is completed, the excess (non-reacted) GSC reagent and other small molecular components such as excess reducing reagent can subsequently be removed by dialysis, tangential flow filtration or size exclusion chromatography.

Both the natural substrate for sialyltransferases, Sia-CMP, and the GSC derivatives are multifunctional molecules that are charged and highly hydrophilic. In addition, they are not stable in solution for extended time periods especially if pH is below 6.0. At such low pH, the CMP activation group necessary for substrate transfer is lost due to acid catalyzed phosphate diester hydrolysis (Yasuhiro Kajihara et al., Chem Eur J 2011, 17, 7645-7655. Selective modification and isolation of GSC and Sia-CMP derivatives thus require careful control of pH, as well as fast and efficient isolation methods, in order to avoid CMP-hydrolysis. In some embodiments, large half-life extending moieties are conjugated to GSC using reductive amination chemistry. Arylaldehydes, such as benzaldhyde modified half-life extending moieties have been found optimal for this type of modification, as they efficiently can react with GSC under reductive amination conditions.

As GSC may undergo hydrolysis in acid media, it is important to maintain a near neutral or slightly basic environment during the coupling to HEP-benzaldehydes. HEP polymers and GSC are both highly water soluble and aqueous buffer systems are therefore preferable for maintaining pH at a near neutral level. A number of both organic and inorganic buffers may be used, however, the buffer components should preferably not be reactive under reductive amination conditions. This exclude for instance organic buffer systems containing primary and—to lesser extend—secondary amino groups. The skilled person will know which buffers are suitable and which are not. Some examples of suitable buffers are shown in table 1 below:

TABLE 1
Buffers

| Common | pKa at | Buffer | |
|---|---|---|---|
| Name | 25° C. | Range | Full Compound Name |
| Bicine | 8.35 | 7.6-9.0 | N,N-bis(2-hydroxyethyl)glycine |
| Hepes | 7.48 | 6.8-8.2 | 4-2-hydroxyethyl-1-piperazineethane- |
| | | | sulfonic acid |
| TES | 7.40 | 6.8-8.2 | 2-{[tris(hydroxymethyl)methyl]amino} |
| | | | ethanesulfonic acid |
| MOPS | 7.20 | 6.5-7.9 | 3-(N-morpholino)propanesulfonic acid |
| PIPES | 6.76 | 6.1-7.5 | piperazine-N,N′-bis(2-ethanesulfonic acid) |
| MES | 6.15 | 5.5-6.7 | 2-(N-morpholino)ethanesulfonic acid |

By applying this method, GSC reagents modified with half-life extending moieties, having isomer free stable linkages can efficient be prepared, and isolated in a simple process that minimize the chance for hydrolysis of the CMP activation group. By reacting either of said compounds with each other a HEP-GSC conjugate comprising a 4-methylbenzoyl sublinker moiety may be created.

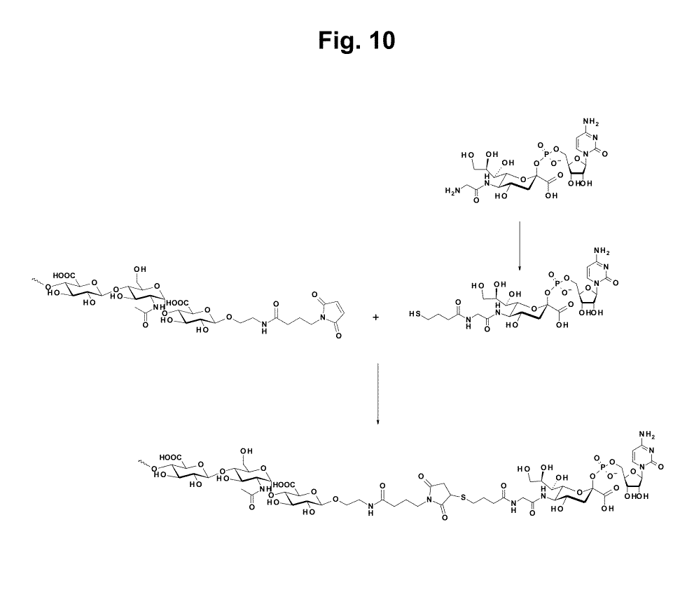

GSC may also be reacted with thiobutyrolactone, thereby creating a thiol modified GSC molecule (GSC-SH). As demonstrated in the present invention, such reagents may be reacted with maleimide functionalized HEP polymers to form HEP-GSC reagents. This synthesis route is depicted in FIG. 10. The resulting product has a linkage structure comprising succinimide.

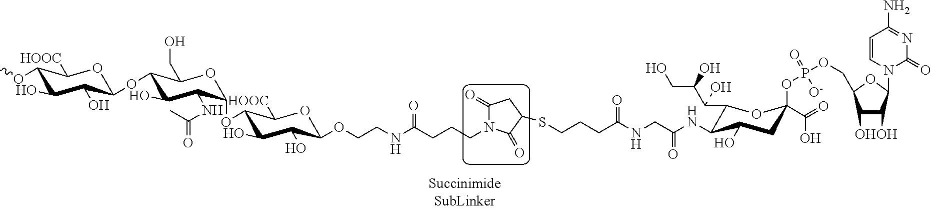

However, succinimide based (sub)linkages may undergo hydrolytic ring opening inter alia when the modified GSC reagent is stored in aqueous solution for extended time periods and while the linkage may remain intact, the ring opening reaction will add undesirable heterogeneity in form of regio- and stereo-isomers.

### Methods of Glycoconjugation

Conjugation of a HEP-GSC conjugate with a (poly)-peptide may be carried out via a glycan present on residues in the (poly)-peptide backbone. This form of conjugation is also referred to as glycoconjugation.

Methods for glycoconjugation of HEP polymers include galactose oxidase based conjugation (WO2005014035) and periodate based conjugation (WO2008025856). Methods based on sialyltransferase have over the years proven to be mild and highly selective for modifying N-glycans or O-glcyans on blood coagulation factors, such as coagulation factor FVII.

In contrast to conjugation methods based on cysteine alkylations, lysine acylations and similar conjugations involving amino acids in the protein backbone, conjugation via glycans is an appealing way of attaching larger structures such as polymers of protein/peptide fragments to bioactive proteins with less disturbance of bioactivity. This is because glycans being highly hydrophilic generally tend to be oriented away from the protein surface and out in solution, leaving the binding surfaces that are important for the proteins activity free. The glycan may be naturally occurring or it may be inserted via e.g. insertion of an N-linked glycan using methods well known in the art.

GSC is a sialic acid derivative that can be transferred to glycoproteins by the use of sialyltransferases. It can be selectively modified with substituents such as PEG on the glycyl amino group and still be enzymatically transferred to glycoproteins by use of sialyltransferases. GSC can be efficiently prepared by an enzymatic process in large scale (WO07056191).

### Sialyltransferases

Sialyltransferases are a class of glycosyltransferases that transfer sialic acid from naturally activated sialic acid (Sia)—CMP (cytidine monophosphate) compounds to galactosyl-moieties on e.g. proteins. Many sialyltransferases (ST3GalIII, ST3GalI, ST6GalNAcI) are capable of transfer of sialic acid—CMP (Sia-CMP) derivatives that have been modified on the C5 acetamido group inter alia with large groups such as 40 kDa PEG (WO03031464). An extensive, but non-limited list of relevant sialyltransferases that can be used with the current invention is disclosed in WO2006094810, which is hereby incorporated by reference in its entirety.

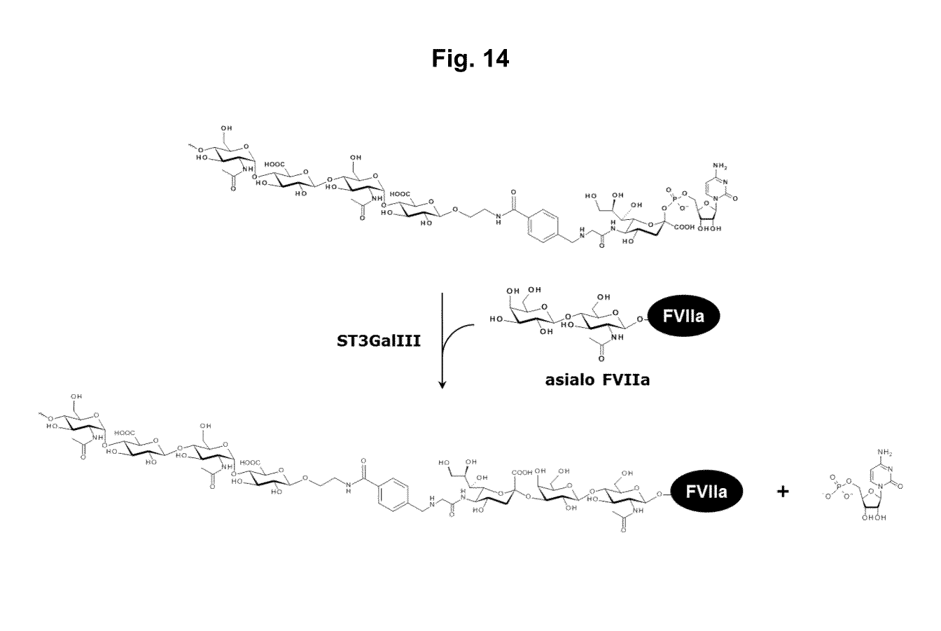

In some embodiments, terminal sialic acids on glycoproteins can be removed by sialidase treatment to provide asialo glycoproteins. Asialo glycoproteins and GSC modified with the half-life extending moiety together will act as substrates for sialyltransferases. The product of the reaction is a glycoprotein conjugate having the half-life extending moiety linked via an intact glycosyl linking group—in this case an intact sialic acid linker group. A reaction scheme wherein an asialo Factor VII glycoprotein is reacted with HEP-GSC in the presence of sialyltransferase is shown in FIG. 14.

### Properties of FVII-HEP Conjugates

In some embodiments, the conjugates described herein have various advantages. For example, the conjugate may show one of more of the following advantages when compared to a suitable control Factor VII molecule.
improved circulating half-life in vivo,
improved mean residence time in vivo
improved biodegradability in vivo
improved biological activity when measured in a proteolysis assay, such as an in vitro proteolysis assay as described herein,
improved biological activity when measured in a clotting assay,
improved biological activity when measured in an in vitro hydrolysis assay as described herein,
improved biological activity when measured in a tissue factor binding assay
improved biological activity when measured in a thrombin generating assay
improved ability to generate Factor Xa.

The conjugate may show an improvement in any biological activity of Factor VII as described herein and this may be measured using any assay or method as described herein, such as the methods described above in relation to the activity of Factor VII.

Advantages may be seen when a conjugate of the invention, i.e. a conjugate of interest, is compared to a suitable control Factor VII molecule. The control molecule may be, for example, an unconjugated Factor VII polypeptide or a conjugated Factor VII polypeptide. The conjugated control may be a FVIIa polypeptide conjugated to a water soluble polymer, or a FVIIa polypeptide chemically linked to a protein.

A conjugated Factor VII control may be a Factor VII polypeptide that is conjugated to a chemical moiety (being protein or water soluble polymer) of a similar size as the heparosan molecule in the conjugate of interest. The water-soluble polymer can for example be polyethylene glycol (PEG), branched PEG, dextran, poly(l-hydroxymethylethylene hydroxymethylformal), 2-methacryloyloxy-2′-ethyltrimethylammoniumphosphate (MPC).

The Factor VII polypeptide in the control Factor VII molecule is preferably the same Factor VII polypeptide that is present in the conjugate of interest. For example, the control Factor VII molecule may have the same amino acid sequence as the Factor VII polypeptide in the conjugate of interest. The control Factor VII may be the same glycosylation pattern as the Factor VII polypeptide in the conjugate of interest.

For example, where the conjugate comprises Factor VII having an additional cysteine at position 407 and the heparosan polymer is attached to that additional cysteine, then the control Factor VII molecule is preferably the same Factor VII molecule having an additional cysteine at position 407, but having no heparosan attached.

Where the activity being compared is the circulating half-life, the control being used for comparison may be a suitable Factor VII conjugated molecule as described above. The conjugate of the invention preferably shows an improvement in circulating half-life, or in mean residence time when compared to a suitable control.

Where the activity being compared is a biological activity of Factor VII, such as clotting activity or proteolysis, the control is preferably a suitable Factor VII polypeptide conjugated to a water soluble polymer of comparable size to the heparosan conjugate of the current invention.

The conjugate may not retain the level of biological activity seen in Factor VII that is not modified by the addition of heparosan. Preferably the conjugate of the invention retains as much of the biological activity of unconjugated Factor VII as possible. For example, the conjugate may retain at least 15%, at least 20%, at least 25%, at least 30%, at least 35%, at least 40%, at least 45%, at least 50% or at least 60% of the biological activity of an unconjugated Factor VII control. As discussed above, the control may be a Factor VII molecule having the same amino acid sequence as the Factor VII polypeptide in the conjugate, but lacking heparosan. The conjugate may, however, show an improvement in biological activity when compared to a suitable control. The biological activity here may be any biological activity of Factor VII as described herein such as clotting activity or proteolysis activity.

An improved biological activity when compared to a suitable control as described herein may be any measurable or statistically significant increase in a biological activity. The biological activity may be any biological activity of Factor VII as described herein, such as clotting activity, proteolytic activity. The increase may be, for example, an increase of at least 5%, at least 10%, at least 15%, at least 20%, at least 25%, at least 30%, at least 35%, at least 40%, at least 45%, at least 50%, at least 55%, at least 60%, at least 70% or more in the relevant biological activity when compared to the same activity in a suitable control.

An advantage of the conjugates of the invention is that heparosan polymers are enzymatically biodegradable. A conjugate of the invention is therefore preferably enzymatically degradable in vivo and/or in vitro.

An advantage of the conjugates of the invention may be that a heparosan polymer linked to Factor VII may reduce or not create inter-assay variability in aPTT-based assays.

### Compositions and Formulations

In another aspect, the present invention provides compositions and formulations comprising conjugates described herein. For example, the invention provides a pharmaceutical composition comprising one or more conjugates, formulated together with a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible.

In some embodiments, pharmaceutically acceptable carriers comprise aqueous carriers or diluents. Examples of suitable aqueous carriers that may be employed in the pharmaceutical compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. In many cases, it will be preferable to include isotonic agents, for example, sugars, polyalcohols such as mannitol, sorbitol, or sodium chloride in the composition.

The pharmaceutical compositions are primarily intended for parenteral administration for prophylactic and/or therapeutic treatment. Preferably, the pharmaceutical compositions are administered parenterally, i.e., intravenously, subcutaneously, or intramuscularly, or it may be administered by continuous or pulsatile infusion. The compositions for parenteral administration comprise the Factor VII conjugate of the invention in combination with, preferably dissolved in, a pharmaceutically acceptable carrier, preferably an aqueous carrier. A variety of aqueous carriers may be used, such as water, buffered water, 0.4% saline, 0.3% glycine and the like. The Factor VII conjugate of the invention can also be formulated into liposome preparations for delivery or targeting to the sites of injury. Liposome preparations are generally described in, e.g., U.S. Pat. No. 4,837,028, U.S. Pat. No. 4,501,728 and U.S. Pat. No. 4,975,282. The compositions may be sterilised by conventional, well-known sterilisation techniques. The resulting aqueous solutions may be packaged for use or filtered under aseptic conditions and lyophilised, the lyophilised preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc.

The concentration of Factor VII conjugate in these formulations can vary widely, i.e., from less than about 0.5% by weight, usually at or at least about 1% by weight to as much as 15 or 20% by weight and will be selected primarily by fluid volumes, viscosities, etc., in accordance with the particular mode of administration selected. Thus, a typical pharmaceutical composition for intravenous infusion can be made up to contain 250 ml of sterile Ringer's solution and 10 mg of the Factor VII conjugate. Actual methods for preparing parenterally administrable compositions will be known or apparent to those skilled in the art and are described in more detail in, for example, Remington's Pharmaceutical Sciences, 18th ed., Mack Publishing Company, Easton, Pa. (1990).

Therapeutic compositions typically must be sterile and stable under the conditions of manufacture and storage. The composition can be formulated as a solution, microemulsion, liposome, or other ordered structure suitable to high drug concentration.

Sterile injectable solutions can be prepared by incorporating conjugates as described herein in the required amount in an appropriate solvent with one or a combination of ingredients enumerated above, as required, followed by sterilization microfiltration. Generally, dispersions are prepared by incorporating the active agent into a sterile vehicle that contains a basic dispersion medium and the required other ingredients from those enumerated above. The composition should be sterile and should be fluid to the extent that easy syringability exists. It should be stable under the conditions of manufacture and storage and may be preserved against the contaminating action of microorganisms such as bacteria and fungi. In the case of sterile powders for the preparation of sterile injectable solutions, the preferred methods of preparation are vacuum drying and freeze-drying (lyophilization) that yield a powder of the active agent plus any additional desired ingredient from a previously sterile-filtered solution thereof.

The conjugate may be used in conjunction with a solvent or dispersion medium containing, for example, water, ethanol, polyol (for example, glycerol, propylene glycol, and liquid poly[ethylene glycol], and the like), suitable mixtures thereof, vegetable oils, and combinations thereof.

The proper fluidity of the conjugate may be maintained, for example, by the use of a coating such as lecithin, by the maintenance of the required particle size in the case of dispersion, and/or by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin.

Sterile injectable solutions may be prepared by incorporating conjugates as described herein in the required amount in an appropriate solvent with one or a combination of ingredients enumerated above, as required, followed by filtered sterilization. Generally, dispersions are prepared by incorporating the heparosan conjugate into a sterile carrier that contains a basic dispersion medium and the required other ingredients from those enumerated above. In the case of sterile powders for the preparation of sterile injectable solutions, the methods of preparation may include vacuum drying, spray drying, spray freezing and freeze-drying that yields a powder of the active ingredient (i.e., the heparosan conjugate) plus any additional desired ingredient from a previously sterile-filtered solution thereof.

Compositions may be formulated in dosage unit form for ease of administration and uniformity of dosage. Dosage unit form as used herein refers to physically discrete units suited as unitary dosages for the subjects to be treated; each unit containing a predetermined quantity of conjugate calculated to produce the desired therapeutic effect. The specification for the dosage unit forms of the presently claimed and disclosed inventions) are dictated by and directly dependent on (a) the unique characteristics of the heparosan conjugate and the particular therapeutic effect to be achieved, and (b) the limitations inherent in the art of compounding such a therapeutic compound for the treatment of a selected condition in a subject.

Pharmaceutical compositions as described herein may comprise additional active ingredients asin addition to a conjugate as described herein. For example, a pharmaceutical composition may comprise additional therapeutic or prophylactic agents. For example, where a pharmaceutical composition of the invention is intended for use in the treatment of a bleeding disorder, it may additionally comprise one or more agents intended to reduce the symptoms of the bleeding disorder. For example, the composition may comprise one or more additional clotting factors. The composition may comprise one or more other components intended to improve the condition of the patient. For example, where the composition is intended for use in the treatment of patients suffering from unwanted bleeding such as patients undergoing surgery or patients suffering from trauma, the composition may comprise one or more analgesic, anaesthetic, immunosuppressant or anti-inflammatory agents.

The composition may be formulated for use in a particular method or for administration by a particular route. A conjugate or composition of the invention may be administered parenterally, intraperitoneally, intraspinally, intravenously, intramuscularly, intravaginally, subcutaneously, intranasally, rectally, or intracerebrally.

An advantageous property of the Factor VII polypeptide and heparosan polymer conjugate, of the invention, is where the polymer has a polymer size around in the range of 13-65 kDa (e.g.13-55 kDa, 25-55 kDa, 25-50 kDa, 25-45 kDa, 30-45 kDa or 38-42 kDa) this may allow for an in vivo useful half-life or mean residence time while also having a suitable viscosity in liquid solution.

### Uses of the Conjugates

A conjugate of the invention may be administered to an individual in need thereof in order to deliver Factor VII to that individual. The individual may be any individual in need of Factor VII.

The Factor VII conjugates described herein may be used to control bleeding disorders which may be caused by, for example, clotting factor deficiencies (e.g. haemophilia A and B or deficiency of coagulation factors XI or VII) or clotting factor inhibitors, or they may be used to control excessive bleeding occurring in subjects with a normally functioning blood clotting cascade (no clotting factor deficiencies or inhibitors against any of the coagulation factors). The bleeding may be caused by a defective platelet function, thrombocytopenia or von Willebrand's disease. They may also be seen in subjects in whom an increased fibrinolytic activity has been induced by various stimuli.

For treatment in connection with deliberate interventions, the Factor VII conjugates of the invention will typically be administered within about 24 hours prior to performing the intervention, and for as much as 7 days or more thereafter. Administration as a coagulant can be by a variety of routes as described herein.

The dose of the Factor VII conjugates delivers from about 0.05 mg to 500 mg of the Factor VII polypeptide/day, preferably from about 1 mg to 200 mg/day, and more preferably from about 10 mg to about 175 mg/day for a 70 kg subject as loading and maintenance doses, depending on the weight of the subject and the severity of the condition. A suitable dose may also be adjusted for a particular conjugate of the invention based on the properties of that conjugate, including its in vivo half-life or mean residence time and its biological activity. For example, conjugates having a longer half-life may be administered in reduced dosages and/or compositions having reduced activity compared to wild-type Factor VII may be administered in increased dosages.

The compositions containing the Factor VII conjugates of the present invention can be administered for prophylactic and/or therapeutic treatments. In therapeutic applications, compositions are administered to a subject already suffering from a disease, such as any bleeding disorder as described above, in an amount sufficient to cure, alleviate or partially arrest the disease and its complications. An amount adequate to accomplish this is defined as “therapeutically effective amount”. As will be understood by the person skilled in the art amounts effective for this purpose will depend on the severity of the disease or injury as well as the weight and general state of the subject. In general, however, the effective delivery amount will range from about 0.05 mg up to about 500 mg of the Factor VII polypeptide per day for a 70 kg subject, with dosages of from about 1.0 mg to about 200 mg of the Factor VII being delivered per day being more commonly used.

The conjugates described herein may generally be employed in serious disease or injury states, that is, life threatening or potentially life threatening situations. In such cases, in view of the minimisation of extraneous substances and general lack of immunogenicity of human Factor VII polypeptide variants in humans, it may be felt desirable by the treating physician to administer a substantial excess of these Factor VII conjugate compositions. In prophylactic applications, compositions containing the Factor VII conjugate of the invention are administered to a subject susceptible to or otherwise at risk of a disease state or injury to enhance the subject's own coagulative capability. Such an amount is defined to be a “prophylactically effective dose.” In prophylactic applications, the precise amounts of Factor VII polypeptide being delivered once again depend on the subject's state of health and weight, but the dose generally ranges from about 0.05 mg to about 500 mg per day for a 70-kilogram subject, more commonly from about 1.0 mg to about 200 mg per day for a 70-kilogram subject.

Single or multiple administrations of the compositions can be carried out with dose levels and patterns being selected by the treating physician. For ambulatory subjects requiring daily maintenance levels, the Factor VII polypeptide conjugates may be administered by continuous infusion using e.g. a portable pump system.

Local delivery of a Factor VII conjugate of the present invention, such as, for example, topical application may be carried out, for example, by means of a spray, perfusion, double balloon catheters, stent, incorporated into vascular grafts or stents, hydrogels used to coat balloon catheters, or other well established methods. In any event, the pharmaceutical compositions should provide a quantity of Factor VII conjugate sufficient to effectively treat the subject.

The present invention is further illustrated by the following examples which, however, are not to be construed as limiting the scope of protection. The features disclosed in the foregoing description and in the following examples may, both separately and in any combination thereof, be material for realising the invention in diverse forms thereof.

Dotted lines in structure formulas denotes open valence bond (i.e. bonds that connect the structures to other chemical moieties).

### DEFINITIONS

Unless defined otherwise, all technical and scientific terms used herein generally have the same meaning as commonly understood by one of ordinary skill in the art to which this, invention belongs.

The term “coagulopathy”, as used herein, refers to an increased haemorrhagic tendency which may be caused by any qualitative or quantitative deficiency of any pro-coagulative component of the normal coagulation cascade, or any upregulation of fibrinolysis. Such coagulopathies may be congenital and/or acquired and/or iatrogenic and are identified by a person skilled in the art.

The term “glycan” refers to the entire oligosaccharide structure that is covalently linked to a single amino acid residue. Glycans are normally N-linked or O-linked, e.g., glycans are linked to an asparagine residue (N-linked glycosylation) or a serine or threonine residue (O-linked glycosylation). N-linked oligosaccharide chains may be multi-antennary, such as, e.g., bi-, tri, or tetra-antennary and most often contain a core structure of Man3-GlcNAc-GlcNAc-. Both N-glycans and O-glycans are attached to proteins by the cells producing the protein. The cellular N-glycosylation machinery recognizes and glycosylates N-glycosylation consensus motifs (N—X-SIT motifs) in the amino acid chain, as the nascent protein is translocated from the ribosome to the endoplasmic reticulum (Kiely et al. 1976; Glabe et al. 1980). Some glycoproteins, when produced in a human in situ, have a glycan structure with terminal, or “capping”, sialic acid residues, i.e., the terminal sugar of each antenna is N-acetylneuraminic acid linked to galactose via an a2->3 or a2->6 linkage. Other glycoproteins have glycans end-capped with other sugar residues. When produced in other circumstances, however, glycoproteins may contain oligosaccharide chains having different terminal structures on one or more of their antennae, such as, e.g., containing N-glycolylneuraminic acid (NeuSGc) residues or containing a terminal N-acetylgalactosamine (GaINAc) residue in place of galactose.

The term “sialic acid” refers to any member of a family of nine-carbon carboxylated sugars. The most common member of the sialic acid family is N-acetylneuraminic acid (2-keto-5-acetamido-3,5-dideoxy-D-glycero-D-galactononulopyranos-1-onic acid (often abbreviated as NeuSAc, NeuAc, NeuNAc, or NANA). A second member of the family is N-glycolyl-neuraminic acid (NeuSGc or NeuGc), in which the N-acetyl group of NeuNAc is hydroxylated. A third sialic acid family member is 2-keto-3-deoxy-nonulosonic acid (KDN) (Nadano et al. (1986) J. Biol. Chem. 261: 11550-11557; Kanamori et al., J. Biol. Chem. 265: 21811-21819 (1990)). Also included are 9-substituted sialic acids such as a 9-O-C1-C6 acyl-Neu5Ac like 9-O-lactylNeu5Ac or 9-O-acetyl-Neu5Ac. The synthesis and use of sialic acid compounds in a sialylation procedure is disclosed in international application WO92/16640, published Oct. 1, 1992.

The term “sialic acid derivative” refers to sialic acids as defined above that are modified with one or more chemical moieties. The modifying group may for example be alkyl groups such as methyl groups, azido- and fluoro groups, or functional groups such as amino or thiol groups that can function as handles for attaching other chemical moieties. Examples include 9-deoxy-9-fluoro-Neu5Ac and 9-azido-9-deoxy-Neu5Ac. The term also encompasses sialic acids that lack one of more functional groups such as the carboxyl group or one or more of the hydroxyl groups. Derivatives where the carboxyl group is replaced with a carboxamide group or an ester group are also encompassed by the term. The term also refers to sialic acids where one or more hydroxyl groups have been oxidized to carbonyl groups. Furthermore the term refers to sialic acids that lack the C9 carbon atom or both the C9-C8 carbon chain for example after oxidative treatment with periodate.

Glycyl sialic acid is a sialic acid derivative according to the definition above, where the N-acetyl group of NeuNAc is replaced with a glycyl group also known as an amino acetyl group. Glycyl sialic acid may be represented with the following structure:

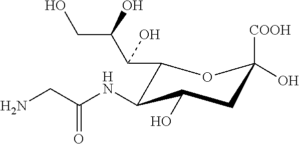

The term “CMP-activated” sialic acid or sialic acid derivatives refer to a sugar nucleotide containing a sialic acid moiety and a cytidine monophosphate (CMP).

In the present description, the term “glycyl sialic acid cytidine monophosphate” is used for describing GSC, and is a synonym for alternative naming of same CMP activated glycyl sialic acid. Alternative naming include CMP-5′-glycyl sialic acid, cytidine-5′-monophospho-N-glycylneuraminic acid, cytidine-5′-monophospho-N-glycyl sialic acid. The term “sialic acid” refers to any member of a family of nine-carbon carboxylated sugars. The most common member of the sialic acid family is N-acetylneuraminic acid (2-keto-5-acetamido-3,5-dideoxy-D-glycero-D-galactononulopyranos-1-onic acid (often abbreviated as NeuSAc, NeuAc, or NANA). A second member of the family is N-glycolyl-neuraminic acid (Neu5Gc or NeuGc), in which the N-acetyl group of NeuAc is hydroxylated. A third sialic acid family member is 2-keto-3-deoxy-nonulosonic acid (KDN) (Nadano et al. (1986) J. Bioi. Chem. 261: 11550-11557; Kanamori et aI., J. Bioi. Chern. 265: 21811-21819 (1990)). Also included are 9-substituted sialic acids such as a 9-O-C1-C6 acyl-NeuSAc like 9-O-lactylNeuSAc or 9-O-acetyl-NeuSAc, 9-deoxy-9-fiuoro-NeuSAc and 9-azido-9-deoxy-NeuSAc. The synthesis and use of sialic acid compounds in a sialylation procedure is disclosed in international application WO92/16640, published Oct. 1, 1992.

The term “intact glycosyl linking group” refers to a linking group that is derived from a glycosyl moiety in which the saccharide monomer interposed between and covalently attached to the polypeptide and the HEP moiety is not degraded, e.g., oxidized, e.g., by sodium metaperiodate during conjugate formation. “Intact glycosyl linking groups” may be derived from a naturally occurring oligosaccharide by addition of glycosyl unites or removal of one or more glycosyl unit from a parent saccharide structure.

The term “asialo glycoprotein” is intended to include glycoproteins wherein one or more terminal sialic acid residues have been removed, e.g., by treatment with a sialidase or by chemical treatment, exposing at least one galactose or N-acetylgalactosamine residue from the underlying “layer” of galactose or N-acetylgalactosamine (“exposed galactose residue”).

Dotted lines in structure formulas denotes open valence bond (i.e. bonds that connect the structures to other chemical moieties).

### EXAMPLES

Abbreviations used in the examples:

CMP: Cytidine monophosphate

EDTA: Ethylenediaminetetraacetic acid

Gla: Gamma-carboxyglutamic acid

GlcUA: Glucuronic acid

GlcNAc: N-acetylglucosamine

Grx2: Glutaredoxin II

GSC: Glycyl sialic acid cytidine monophosphate

GSC-SH: [(4-mercaptobutanoyl)glycyl]sialic acid cytidine monophosphate

GSH: Glutathione

GSSG: Glutathione disulfide

HEP: HEParosan polymer

HEP-GSC: GSC-functionalized heparosan polymers

HEP-[C]-FVIIa407C: HEParosan conjugated via cysteine to FVIIa407C.

HEP-[N]-FVIIa: HEParosan conjugated via N-glycan to FVIIa.

HEPES: 2-[4-(2-hydroxyethyl)piperazin-1-yl]ethanesulfonic acid

His: Histidine

PmHS1: Pasteurella mutocida Heparosan Synthase I

sTF: soluble Tissue Factor

TCEP: Tris(2-carboxyethyl)phosphine

UDP: Uridine diphosphate

### Quantification Method

The conjugates of the invention were analysed for purity by HPLC. HPLC was also used to quantify amount of isolated conjugate based on a FVIIa reference molecule. Samples were analysed either in non-reduced or reduced form. A Zorbax 300SB-C3 column (4.6×50 mm; 3.5 μm Agilent, Cat. No.: 865973-909) was used. Column was operated at 30° C. 5 μg sample was injected, and column was eluted with a water (A)—acetonitrile (B) solvent system containing 0.1% trifluoroacetic acid. The gradient program was as follows: 0 min (25% B); 4 min (25% B); 14 min (46% B); 35 min (52% B); 40 min (90% B); 40.1 min (25% B). Reduced samples were prepared by adding 10 ul TCEP/formic acid solution (70 mM tris(2-carboxyethyl)phosphine and 10% formic acid in water) to 25 μl/30 ug FVIIa (or conjugate). Reactions were left for 10 minutes at 70° C., before analysis on HPLC (5 μl injection). Heparosan polymers were quantified by carbazol assay according to the method by Bitter T, Muir H M. Anal Biochem 1962 October; 4:330-4.

### SDS-PAGE Analysis

SDS PAGE analysis was performed using precast Nupage 7% tris-acetate gel, NuPage tris-acetate SDS running buffer and NuPage LDS sample buffer all from Invitrogen. Samples were denaturized (70° C. for 10 min.) before analysis. HiMark HMW (Invitrogen) was used as standard. Electrophoresis was run in XCell Surelock Complete with power station (Invitrogen) for 80 min at 150 V, 120 mA. Gels were stained using SimplyBlue SafeStain from Invitrogen.

### Example 1

### Synthesis of HEP-Maleimide and HEP-Aldehyde Polymers

Maleimide and aldehyde functionalized HEP polymers of defined size are prepared by an enzymatic (PmHS1) polymerization reaction using the two sugar nucleotides UDP-GlcNAc and UDP-GlcUA. A priming trisaccharide (GlcUA-GlcNAc-GlcUA)NH₂ is used for initiating the reaction, and polymerization is run until depletion of sugar nucleotide building blocks. The terminal amine (originating from the primer) is then functionalized with suitable reactive groups, in this case either a maleimide functionality designed for conjugation to free cysteines and thioGSC derivatives, or a benzaldehyde functionality designed for reductive amination chemistry to GSC. Size of HEP polymers can be pre-determined by variation in sugar nucleotide: primer stoichiometry. The technique is described in detail in US 2010/0036001.

The trisaccharide primer is synthesised as follows:

### Step 1: Synthesis of (2-Fmoc-amino)ethyl 2,3,4-tri-O-acetyl-β-D-glucuronic acid methyl ester

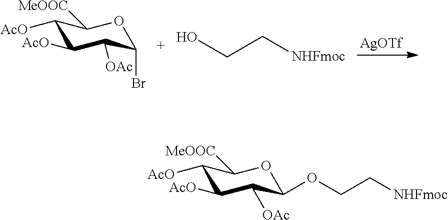

Powdered molecular sieves (1.18 g, 4 Å) were heated at 110° C. in a 50 ml round bottom flask fitted with a magnetic stir bar overnight, flushed with argon, and allowed to cool to room temperature. 900 mg (2.19 mmol) aceto-bromo-β-D-glucuronic acid methyl ester and 748.5 mg (2.64 mmol, 1.2 eq) 2-(Fmoc-amino)ethanol were added under argon, followed by 28 ml dichloromethane. The suspension was stirred for 15 minutes at room temperature and then cooled on an ice/NaCl-slurry for 30 minutes. A white precipitate formed during the cooling process. 676.3 mg (2.63 mmol, 1.2 eq) silver trifluoromethanesulfonate (AgOTf) was added in 3 portions over a period of ˜5 minutes. After 20 minutes the ice-bath was removed. The previously noted white precipitate started dissolving, while at the same time a grey precipitate started to form. The reaction was stirred overnight at room temperature and then quenched by addition of 190 μL triethylamine (2.63 mmol, 1.2 eq). After filtration through a thin Celite 521 pad (˜0.1-0.2 cm deep), and subsequent washing of the filter cake with 20 ml dichloromethane, the combined filtrates were diluted with dichloromethane to 150 ml. The organic phase was washed with 5% NaHCO₃ (1×50 mL) and water (1×50 mL), then dried over magnesium sulfate and filtered. The filtrate was concentrated in vacuo on a rotary evaporator (≦40° C. water bath) to dryness and then re-dissolved in 2 mL dichloromethane. The solution was injected onto a VersaPak silica gel flash column (23×110 mm, 23 g) and the product eluted with 50% ethyl acetate in hexanes. The product-containing fractions were identified by TLC (ethyl acetate:hexanes, 1:1), and concentrated in vacuo on a rotary evaporator (≦40° C. water bath) to dryness. Trituration of the obtained residue with ˜10 mL diethyl ether yielded the title material as a white crystalline foam. Yield: 293 mg (0.49 mmol, 22.4%).

### Step 2: Synthesis of (2-Fmoc-amino)ethyl β-D-glucuronic acid, sodium salt

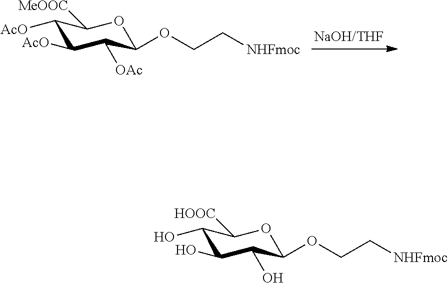

490 mg (0.817 mmol, 1 eq) of (2-Fmoc-amino)ethyl 2,3,4-tri-O-acetyl-β-D-glucuronic acid methyl ester obtained in step 1 was dissolved in 47.5 mL methanol and 2.5 mL (2.45 mmol, 3 eq) of a 1 M NaOH-solution was slowly added under stirring. The reaction was monitored by TLC using 1-butanol:acetic acid:water=1:1:1 as eluent. After TLC showed complete consumption of the methyl ester, the pH of the reaction mixture was lowered to pH 8-9 by addition of 1 N HCl. 204 mg (2.45 mmol, 3 eq) solid NaHCO₃ followed by 241.7 mg (0.899 mmol, 1.1 eq) Fmoc-chloride was then added. When TLC analysis showed completion of reaction, the reaction mixture was diluted with ˜150 mL water, extracted twice with ethyl acetate (2×30 mL), and then concentrated in vacuo over a 40° C. water bath to about 20 mL to remove any remaining organic solvents. The solution was acidified by addition of acetic acid to a content of ˜5% (v:v), and passed through a 5 gram Strata C-18E SPE tube (pre-wetted in methanol, and equilibrated in 5% acetic acid according to manufacturer's instructions). The resin was washed with 5% acetic acid, and the product was eluted with a mixture of 90% methanol with 10% Tris.HCl, pH 7.2 (v:v). After concentration in vacuo (<40° C. water bath) to dryness, the residue was redissolved and the pH was adjusted to pH 7.2 with sodium hydroxide. This solution was used directly as stock solution in the synthesis of (2-Fmoc-amino)ethyl 4-O-(2-deoxy-2-acetamido-α-D-glucopyranosyl)-β-D-glucuronic acid below without further purification.

### Step 3: Synthesis of (2-Fmoc-amino)ethyl 4-O-(2-deoxy-2-acetamido-α-D-glucopyranosyl)-β-D-glucuronic acid, sodium salt

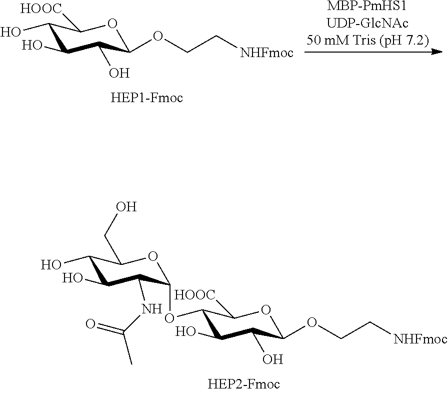

To a solution of 380 mg (2-Fmoc-amino)ethyl β-D-glucuronic acid obtained in step 2 (0.83 mmole, 1 eq) in 100.8 mL water was added 5.6 mL 1 M Tris-HCl, pH 7.2, 5.6 mL 100 mM MnCl₂, and 1.8 g UDP-GlcNAc (2.79 mmole, 3.4 eq). After slow addition of 5.1 mL MBP-PmHS1 enzyme (15.47 mg/mL; 78.9 mg) over ˜1 min, the reaction was left to stir slowly at room temperature until TLC analysis (1-butanol:acetic acid:water=2:1:1) showed nearly complete conversion of starting material. The solution was acidified by addition of 2.8 mL acetic acid to precipitate the spent MBP-PmHS1 and transferred into 50 mL centrifuge bottles. The solution was then centrifuged for 30 min at 10,000 rpm in a JM-12 rotor (˜16,000×g) at room temperature. The supernatant was decanted and added 160 mL methanol. The pellet was extracted 4×25 mL with a solution of water:methanol:acetic acid=45:50:5 (v:v:v). The combined supernatant and extracts were passed through 2 g Strata-SAX tubes (equilibrated in water:methanol:acetic acid=45:50:5 (v:v:v)) to remove any UDP & UDP-GlcNAc (complete removal required 28 grams of resin). The target molecule was unretained and passed through the resin under these conditions; while the more highly charged UDP & UDP-GlcNAc were retained. The combined eluates were concentrated in vacuo (water batch; ≦40° C.), re-dissolved in water, and the pH was adjusted to pH 7.2 using sodium hydroxide. This solution was used directly in the next step without further purification.

### Step 4: Synthesis of (2-Fmoc-amino)ethyl 4-O-(2-deoxy-2-acetamido-4-O-(β-D-glucopyranosyluronic acid)-α-D-glucopyranosyl)-β-D-glucuronic acid, disodium salt

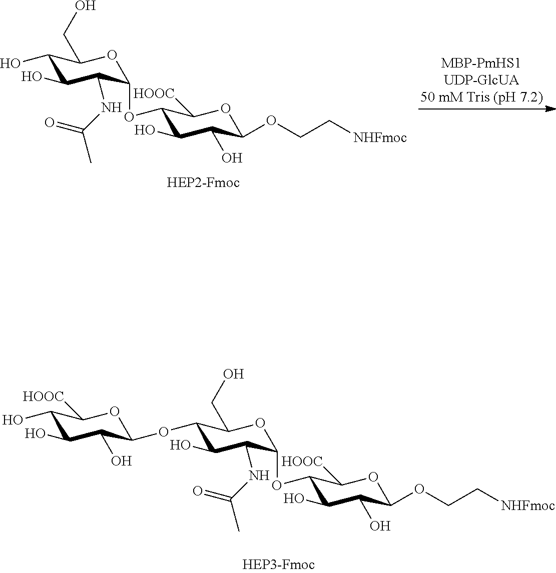

An aqueous solution (38 ml) containing 9 mM (2-Fmoc-amino)ethyl 4-O-(2-deoxy-2-acetamido-α-D-glucopyranosyl)-β-D-glucuronic acid, 30 mM UDP-GlcUA, 50 mM Tris.HCl, and 5 mM MnCl₂ was placed in a spinner flask. Over a period ˜1 min, 9.5 mL MBP-PmHS1 was added dropwise under slow agitation. The reaction mixture was left to stir overnight, after which TLC analysis (eluent: n-BuOH:AcOH:H2O=4:1:1 (v:v:v)) showed complete conversion of the starting material. The reaction mixture was filtered through a 1 μm glass fiber syringe filter, and passed through a 5 gram C18-E SPE tube (equilibrated in water, following manufacturer's instructions). The resin was washed with water, followed by elution of the target molecule with a mixture of 90% aqueous MeOH, 1 mM Tris.HCl, pH 7.2. The eluate was concentrated in vacuo (waterbath <40° C.), then re-dissolved in 25 mL 10 mM Tris.HCl, pH 7.2, and filtered through a 0.2 nm SFCA syringe filter. The filtrate containing the target molecule was further purified by anion exchange chromatography. An Akta Explorer 100 furnished with a 2.6×13 cm Q Sepharose HP column and operated with Unicorn 5.11 software was used. Two buffer systems (buffer A: 10 mM Tris.HCl, pH 7.2 and buffer B: 10 mM Tris.HCl, pH 7.2, 1 M NaCl) were used for elution. The target molecule was eluted using a 0-20% B gradient over 175 min; at a flowrate of 10 ml/min. 10 ml fraction were collected. The fractions containing product were combined, concentrated on a rotary evaporator in vacuo (waterbath <40° C.) to dryness, and used in the next step without further purification.

### Step 5: Synthesis (2-aminoethyl) 4-O-(2-deoxy-2-acetamido-4-O-(β-D-glucopyranosyluronic acid)-α-D-glucopyranosyl)-β-D-glucuronic acid, disodium salt

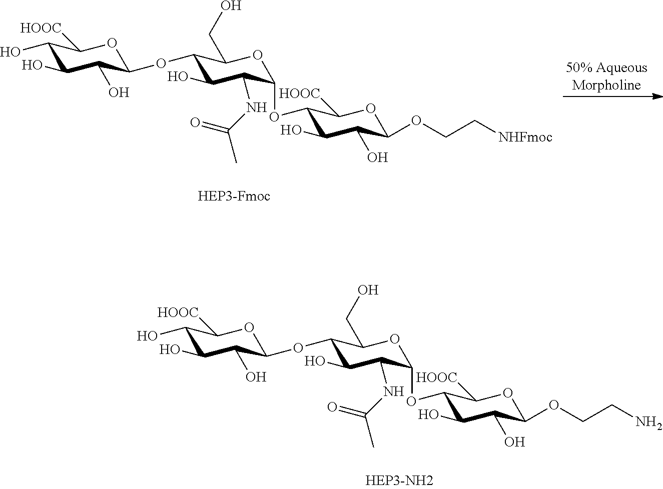

(2-Fmoc-amino)ethyl 4-O-(2-deoxy-2-acetamido-4-O-(β-D-glucopyranosyluronic acid)-α-D-glucopyranosyl)-β-D-glucuronic acid, disodium salt obtained as described in step 4, was dissolved in 4 mL water and cooled on an ice-bath. A volume of 4 mL neat morpholine was added under stirring and the ice bath was removed. Stirring was continued at room temperature, until TLC analysis (n-BuOH:AcOH:H₂O=3:1:1 (v:v:v)) using UV 254 nm detection showed complete consumption of starting material. Reaction was complete within less than 1.5 hrs. The reaction mixture was diluted with ˜50 mL water and extracted three times with 50 mL EtOAc. The aqueous phase containing the target molecule was concentrated on a rotary evaporator in vacuo (waterbath <40° C.) and co-evaporated three times with water. The residue was re-dissolved in 10 mL water and passed through a 1 gram SDB-L SPE column preequilibrated in water. The target passed through the column unretained. The column was washed with 10 mL water and the combined fractions with target were concentrated in vacuo to dryness (water bath; ≦40° C.). The obtained residue was dissolved in 1.5 mL 1 M NaOAc, pH 7.5, filtered through a 0.2 μm spinfilter, and desalted by size-exclusion chromatography over a Sephadex G-10 column (2×75 cm, 235 mL) with water as eluent. Structure of the title material was confirmed by MALDI-TOF MS (matrix: 5 mg/mL ATT; 50% acetonitrile/0.05% trifluoroacetic acid): 636.83 [M+Na⁺]. After lyophilization, the title material was dissolved in water, the pH of the obtained solution was adjusted to pH 7.0-7.5 by addition of sodium hydroxide, and the trisaccharide content was determined by carbazole assay (Bitter T, Muir H M. Anal Biochem 1962 October; 4:330-4). The obtained stock solution was aliquoted and stored at −80° C. in tightly sealed containers until needed. The overall isolated yield of (2-aminoethyl) 4-O-(2-deoxy-2-acetamido-4-O-(6-D-glucopyranosyluronic acid)-α-D-glucopyranosyl)-β-D-glucuronic acid starting from (2-Fmoc-amino)ethyl β-D-glucuronic acid was 210 mg (0.34 mmole, 41%).

Production of Heparosan Polysaccharide with Amine Terminal

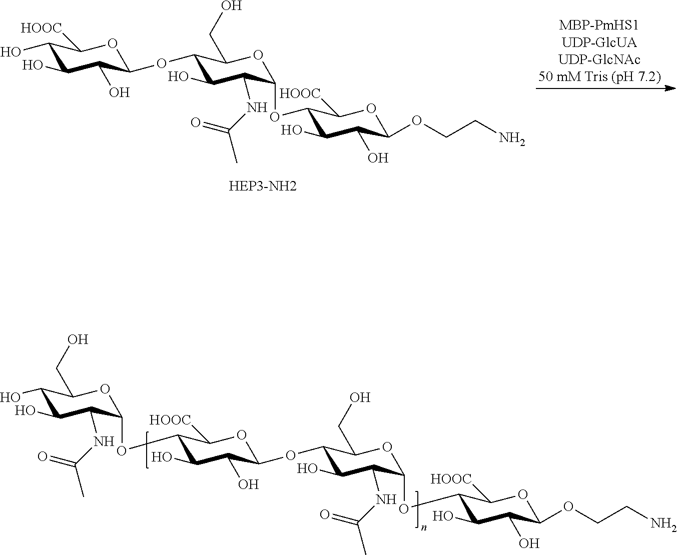

To obtain a heparosan polymer derivative with a free amine group (HEP-NH₂), the Pasteurella multocida heparosan synthase 1 (PmHS1; DeAngelis & White, 2002 J Biol Chem) was used to chemoenzymatically synthesize polymer chains in a parallel fashion in vitro (Sismey-Ragatz et al., 2007 J Biol Chem and U.S. Pat. No. 8,088,604). A fusion of the E. coli maltose-binding protein with PmHS1 was used as the catalyst for elongating the (2-aminoethyl) 4-O-(2-deoxy-2-acetamido-4-O-(6-D-glucopyranosyluronic acid)-α-D-glucopyranosyl)-β-D-glucuronic acid (HEP3-NH₂) obtained in step 5 into longer polymer chains using UDP-GlcNAc and UDP-GlcUA precursors and MnCl₂ catalysis as described in US2010036001.

### Synthesis of HEP-Maleimide and HEP-Benzaldehyde Polymers:

HEP-benzaldehydes can be prepared by reacting amine functionalized HEP polymers with a surplus of N-succinimidyl-4-formylbenzoic acid (Nano Letters (2007) 7(8), pp. 2207-2210) in aqueous neutral solution. The benzaldehyde functionalized polymers may be isolated by ion-exchange chromatography, size exclusion chromatography, or HPLC. HEP-maleimides can be prepared by reacting amine functionalized HEP polymers with a surplus of N-maleimidobutyryloxysuccinimide ester (GMBS; Fujiwara, K., et al. (1988) J Immunol Meth 112, 77-83).

More specifically, to obtain a heparosan polymer derivative for coupling via reductive amination, etc. to accessible amino functionalities on the target drug compound, heparosan-NH₂, was coupled with N-succinimidyl-4-formylbenzoic acid, to form a benzaldehyde-modified heparosan polymer. Basically, in one example, N-succinimidyl-4-formylbenzoic acid (Chem-Impex, Inc) dissolved in dimethyl sulfoxide (11.94 mg in 205 mL) was slowly added to a stirred solution of 62.7 g of 43.8 kDa heparosan polymer-NH₂ dissolved in 380 mL 1M sodium phosphate, pH 7.0, 2180 ml water, and 1040 mL dimethylsulfoxide. The reaction mixture was left to stir at room temperature overnight, followed by alcohol precipitation at ambient temperature. The pellet with product was dissolved in 3 L of 500 mM sodium acetate, pH 6.8, further purified and then concentrated by cross flow filtration. The benzaldehyde or maleimide functionalized polymers may alternatively be isolated by ion-exchange chromatography, size exclusion chromatography, or HPLC.

Any HEP polymer functionalized with a terminal primary amine functionality (HEP-NH₂) may be used in the present examples. Two options are shown below:

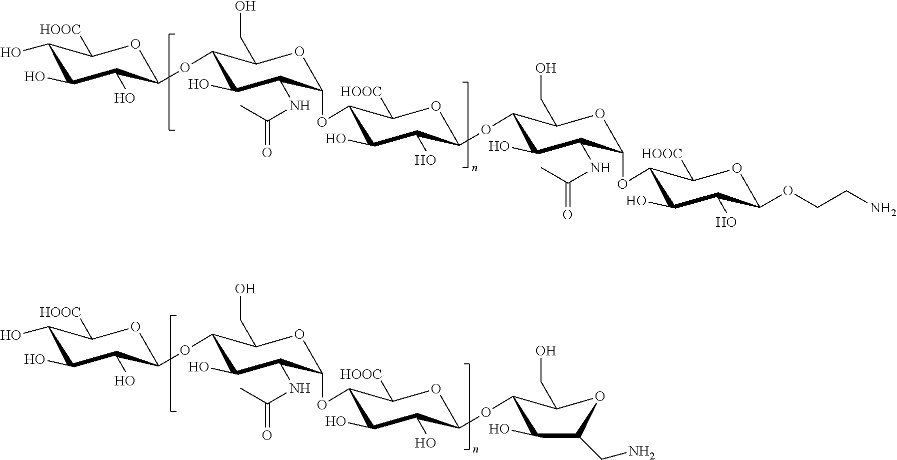

Furthermore the terminal sugar residue in the non-reducing end of the polysaccharide can be either N-acetylglucosamine or glucuronic acid (glucuronic acid is drawn above). Typically a mixture of both is to be expected if equimolar amount of UDP-GlcNAc and UDP-GlcUA has been used in the polymerization reaction. n can be 5-450, such as 50 to 400; 100 to 200; or 150 to 190.

### Example 2

### Selective Reduction of FVIIa407C

FVIIa407C was reduced as described in US 20090041744 using a glutathione based redox buffer system. Non-reduced FVIIa 407C (15.5 mg) was incubated for 17 h at room temperature in a total volume of 41 ml 50 mM Hepes, 100 mM NaCl, 10 mM CaCl₂, pH 7.0 containing 0.5 mM GSH, 15 uM GSSG, 25 mM p-aminobenzamidine and 3 nM Grx2. The reaction mixture was then cooled on ice, and added 8.3 ml 100 mM EDTA solution while keeping pH at 7.0. The entire content was then loaded onto a 5 ml HiTrap Q FF column (Amersham Biosciences, GE Healthcare) equilibrated in buffer A (50 mM Hepes, 100 mM NaCl, 1 mM EDTA, pH 7.0) to capture FVIIa 407C. After wash with buffer A to remove unbound glutathione buffer and Grx2, FVIIa 407C was eluted in one step with buffer B (50 mM Hepes, 100 mM NaCl, 10 mM CaCl₂, pH 7.0). The FVIIa 407C concentration in the eluate was determined by HPLC. 12.6 mg of single cysteine reduced FVIIa407C was isolated in 50 mM Hepes, 100 mM NaCl, 10 mM CaCl₂, pH 7.0.

### Example 3

### Synthesis of 38.8 kDa HEP-[C]-FVIIa407C

Synthesis of 38.8k HEP-[C]-FVIIa 407C: Single cysteine reduced FVIIa 407C (25 mg) was reacted with 38.8K HEP-maleimide (26.8 mg) in 50 mM Hepes, 100 mM NaCl, 10 mM CaCl2, pH 7.0 buffer (8.5 ml) for 22 hours at 5° C. The reaction mixture was then loaded on to a FVIIa specific affinity column (CV=64 ml) modified with a Gla-domain specific antibody and step eluted first with 2 column volumes of buffer A (50 mM Hepes, 100 mM NaCl, 10 mM CaCl2, pH 7.4) then two column volumes of buffer B (50 mM Hepes, 100 mM NaCl, 10 mM EDTA, pH 7.4). The method essentially follows the principle described by Thim, L et al. Biochemistry (1988) 27, 7785-779. The products with unfolded Gla-domain was collected and directly applied to a 3×5 ml HiTrap Q FF ion-exchange column (Amersham Biosciences, GE Healthcare, CV=15 ml) pre-equilibrated with 10 mM His, 100 mM NaCl, pH 7.5. The column was washed with 4 column volumes of 10 mM His, 100 mM NaCl, pH 7.5 and 15 column volumes of 10 mM His, 100 mM NaCl, 10 mM CaCl2, pH 7.5 to eluted unmodified FVIIa 407C. The pH was then lowered to 6.0 with 10 mM His, 100 mM NaCl, 10 mM CaCl2, pH 6.0 (12 column volumes). 38.8k-HEP-[C]-FVIIa407C was eluted with 15 column volumes of a 60% A (10 mM His, 100 mM NaCl, 10 mM CaCl2, pH 6.0) and 40% B (10 mM His, 1 M NaCl, 10 mM CaCl2, pH 6.0) buffer mixture. Fractions containing conjugate were combined, and dialyzed against 10 mM His, 100 mM NaCl, 10 mM CaCl2, pH 6.0 using a Slide-A-Lyzer cassette (Thermo Scientific) with a cut-off of 10kD. The final volume was adjusted to 0.4 mg/ml (8 uM) by addition of 10 mM His, 100 mM NaCl, 10 mM CaCl2, pH 6.0. Yield (16.1 mg, 64%) was determined by quantifying the FVIIa light chain content against a FVIIa standard after TCEP reduction using reverse phase HPLC.

### Example 4

### Synthesis of 65 kDa HEP-[C]FVIIa407C

Single cysteine reduced FVIIa 407C (8 mg) was reacted with 65 kDa HEP-maleimide (42 mg 1:4 ratio) in 50 mM Hepes, 100 mM NaCl, 10 mM CaCl₂, pH 7.0 buffer (8 ml) for 3 hours at room temperature. The reaction mixture was then applied to a FVIIa specific affinity column (CV=24 ml) modified with a Gla-domain specific antibody and step eluted first with buffer A (50 mM Hepes, 100 mM NaCl, 10 mM CaCl₂, pH 7.4) then buffer B (50 mM Hepes, 100 mM NaCl, 10 mM EDTA, pH 7.4). The method essentially follows the principle described by Thim, L et al. Biochemistry (1988) 27, 7785-779. The products with unfolded Gla-domain was collected and directly applied to a HiTrap Q FF ion-exchange column (Amersham Biosciences, GE Healthcare) pre-equilibrated with 10 mM His, 100 mM NaCl, pH 7.5. Unmodified FVIIa 407C was eluted with 5 column volumes of 10 mM His, 100 mM NaCl, 10 mM CaCl2, pH 7.5. The pH was then lowered to 6.0 with 2 column volumes of 10 mM His, 100 mM NaCl, 10 mM CaCl2, pH 6.0. 65 kDa HEP-[C]-FVIIa407C was eluted using a linear gradient. Buffer A (10 mM His, 100 mM NaCl, 10 mM CaCl2, 0.01% Tween80, pH 6.0) and buffer B (10 mM His, 1 M NaCl, 10 mM CaCl2, 0.01% Tween80, pH 6.0) was used for elution. The gradient was 0-100% B buffer over 10 column volumes, at a flow of 0.5 ml/min. The 65 kDa HEP-[C]-FVIIa 407C was eluted in approximately 10 mM histidine, ˜300 mM NaCl, 10 mM CaCl₂, 0.01% Tween80, pH 6.0. Yield and concentration was determined by quantifying the content of FVIIa light chain against a FVIIa standard after TCEP reduction using reverse phase HPLC as described above. A total of 3.10 mg (38%) 65 kDa HEP-[C]-FVIIa 407C conjugate was obtained in a concentration of 0.57 mg/ml in 10 mM His, ˜300 mM NaCl, 10 mM CaCl₂, 0.01% Tween80, pH 6.0. The pure conjugate was diluted to 0.4 mg/ml (8 μM) by ultrafiltration, and buffer exchange into 10 mM histidine, 100 mM NaCl, 10 mM CaCl₂, 0.01% Tween 80, pH 6.0 by dialysis.

### Example 5

### Synthesis of 13 kDa HEP-[C]-FVIIa407C

This conjugate was prepared as described in example 3, using FVIIa 407C (17 mg) and 13 kDa HEP-maleimide (8.5 mg). 7.1 mg (41%) 13 kDa HEP-[C]-FVIIa407C was obtained as a 0.4 mg/ml (8 μM) solution in 10 mM Histidine, 100 mM NaCl, 10 mM CaCl₂, 0.01% Tween 80, pH 6.0.

### Example 6

### Synthesis of 27 kDa HEP-[C]-FVIIa407C

This conjugate was prepared as described in example 3, using FVIIa 407C (15.7 mg) and 27 kDa HEP-maleimide (11.2 mg). 6.9 mg (44%) 27 kDa HEP-[C]-FVIIa407C was obtained as a 0.4 mg/ml (8 uM) solution in 10 mM Histidine, 100 mM NaCl, 10 mM CaCl₂, 0.01% Tween 80, pH 6.0.

### Example 7

### Synthesis of 52 kDa HEP-[C]-FVIIa407C

This conjugate was prepared as described in example 3, using FVIIa 407C (8.3 mg) and 52 kDa HEP-maleimide (27 mg). 6.15 mg (71%) 52 kDa HEP-[C]-FVIIa407C was obtained as a 0.4 mg/ml (8 uM) solution in 10 mM Histidine, 100 mM NaCl, 10 mM CaCl₂, 0.01% Tween 80, pH 6.0.

### Example 8

### Synthesis of 60 kDa HEP-[C]FVIIa407C

This conjugate was prepared as described in example 3, using FVIIa 407C (14.3 mg) and 60 kDa HEP-maleimide (68 mg). 8.60 mg (60%) 60 kDa HEP-[C]-FVIIa407C was obtained as a 0.4 mg/ml (8 μM) solution in 10 mM Histidine, 100 mM NaCl, 10 mM CaCl₂, 0.01% Tween 80, pH 6.0.

### Example 9

### Synthesis of 108 kDa HEP-[C]-FVIIa407C

This conjugate was prepared as described in example 3, using FVIIa 407C (20.0 mg) and 108 kDa HEP-maleimide (174 mg). 3.75 mg (19%) 108 kDa HEP-[C]-FVIIa407C was obtained as a 0.4 mg/ml (8 μM) solution in 10 mM Histidine, 100 mM NaCl, 10 mM CaCl₂, 0.01% Tween 80, pH 6.0.

### Example 10

### Synthesis of 157 kDa HEP-[C]FVIIa407C

This conjugate was prepared as described in example 3, using FVIIa 407C (14.5 mg) and 157 kDa HEP-maleimide (180 mg). 4.93 mg (34%) 157k-HEP-[C]-FVIIa407C was obtained as a 0.3 mg/ml (6 μM) solution in 10 mM Histidine, 100 mM NaCl, 10 mM CaCl₂, 0.01% Tween 80, pH 6.0.

### Example 11

### Synthesis of [(4-mercaptobutanoyl)glycyl]sialic acid cytidine monophosphate (GSC-SH)

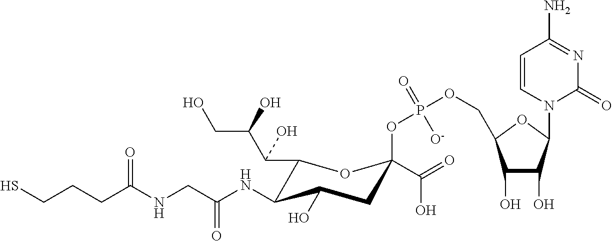

Glycyl sialic acid cytidine monophosphate (200 mg; 0.318 mmol) was dissolved in water (2 ml), and thiobutyrolactone (325 mg; 3.18 mmol) was added. The two phase solution was gently mixed for 21 h at room temperature. The reaction mixture was then diluted with water (10 ml) and applied to a reverse phase HPLC column (C18, 50 mm×200 mm) Column was eluted at a flow rate of 50 ml/min with a gradient system of water (A), acetonitrile (B) and 250 mM ammonium hydrogen carbonate (C) as follows: 0 min (A: 90%, B: 0%, C:10%); 12 min (A: 90%, B: 0%, C:10%); 48 min (A: 70%, B: 20%, C:10%). Fractions (20 ml size) were collected and analysed by LC-MS. Pure fractions were pooled, and passed slowly through a short pad of Dowex 50W×2 (100-200 mesh) resin in sodium form, before lyophilized into dry powder. Content of title material in freeze dried powder was then determined by HPLC using absorbance at 260 nm, and glycylsialic acid cytidine monophosphate as reference material. For the HPLC analysis, a Waters X-Bridge phenyl column (5 μm 4.6 mm×250 mm) and a water acetonitrile system (linear gradient from 0-85% acetonitrile over 30 min containing 0.1% phosphoric acid) was used. Yield: 61.6 mg (26%). LCMS: 732.18 (MH⁺); 427.14 (MH⁺-CMP). Compound was stable for extended periods (>12 months) when stored a −80° C.

### Example 12

### Synthesis of 38.8 kDa HEP-GSC Reagent with Succinimide Linkage

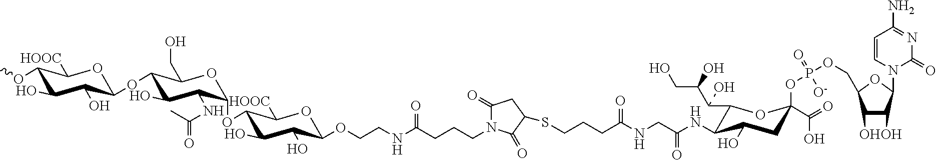

This HEP-GSC reagent was prepared by coupling GSC-SH ([(4-mercaptobutanoyl)glycyl]sialic acid cytidine monophosphate prepared in example 11, with HEP-maleimide in a 1:1 molar ratio as follows: to GSC-SH (0.50 mg) dissolved in 50 mM Hepes, 100 mM NaCl, pH 7.0 (50 μl) was added 26.38 mg of the 38.8 kDa HEP-maleimide dissolved in 50 mM Hepes, 100 mM NaCl, pH 7.0 (1350 μl). The clear solution was left for 2 hours at 25° C. The excess of GSC-SH was removed by dialysis, using a Slide-A-Lyzer cassette (Thermo Scientific) with a cut-off of 10 kDa. The dialysis buffer was 50 mM HEPES, 100 mM NaCl, 10 mM CaCl₂, pH 7.0. The reaction mixture was dialyzed twice for 2.5 hours. The recovered material was used as such, assuming a quantitative reaction between GSC-SH and HEP-maleimide. The HEP-GSC reagent made by this procedure will contain a HEP polymer attached to sialic acid cytidine monophosphate via a succinimide linkage.

### Example 13

### Synthesis of 60 kDa HEP-GSC Reagent with Succinimide Linkage

This molecule was prepared using 60 kDa HEP-maleimide and [(4-mercaptobutanoyl)-glycyl]sialic acid cytidine monophosphate in a similar way as described for 38.8 kDa HEP-GSC above.

### Example 14

### Synthesis of 52 kDa HEP-GSC Reagent with Succinimide Linkage

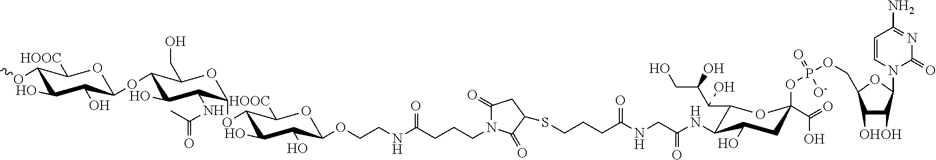

This molecule was prepared using 52 kDa HEP-maleimide and [(4-mercaptobutanoyl)-glycyl]sialic acid cytidine monophosphate in a similar way as described for 38.8 kDa HEP-GSC above.

### Example 15

### Desialylation of FVIIa

FVIIa (28 mg) was added sialidase (Arthrobacter ureafaciens, 200 μl, 0.3 mg/ml, 200 Um′) in 50 mM Hepes, 150 mM NaCl, 10 mM CaCl2, pH 7.0 (18 ml), and left for 1 hour at room temperature. The reaction mixture was then diluted with 50 mM Hepes, 150 mM NaCl, pH 7.0 (30 ml), and cooled on ice. 100 mM EDTA solution (6 ml) was added in small portions. After each addition pH was measured. pH should not exceed 9 or fall below 5.5. The EDTA treated sample was then applied to a 2×5 ml interconnected HiTrap Q FF ion-exchange columns (combined CV=10 ml) pre equilibrated in 50 mM Hepes, 150 mM NaCl, pH 7.0. Sialidase was eluted with 50 mM Hepes, 150 mM NaCl, pH 7.0 (4 CV). Asialo FVIIa was then eluted with 50 mM Hepes, 150 mM NaCl, 10 mM CaCl2, pH 7.0 (10 CV). Yield (24 mg) and concentration (3.0 mg/ml) was determined by quantifying the content of FVIIa light chain against a FVIIa standard after tris(2-carboxyethyl)phosphine reduction using reverse phase HPLC as described previously.

### Example 16

### Synthesis of 52 kDa HEP-[N]-FVIIa with Succinimide Linkage

To asialo FVIIa (7.2 mg) in 50 mM Hepes, 150 mM NaCl, 10 mM CaCl2, pH 7.0 (2.5 ml) was added 52 kDa-HEP-GSC (15.8 mg) from example 14, and rat ST3GalIII enzyme (1 mg; 1.1 unit/mg) in 20 mM Hepes, 120 mM NaCl, 50% glycerol, pH 7.0 (2 ml). The reaction mixture was incubated for 18 hours at 32° C. under slow stirring. A solution of 157 mM CMP-NAN in 50 mM Hepes, 150 mM NaCl, 10 mM CaCl2, pH 7.0 (0.2 ml) was then added, and the reaction was incubated at 32° C. for an additional hour. The reaction mixture was then applied to a FVIIa specific affinity column (CV=25 ml) modified with a Gla-domain specific antibody and step eluted first with 2 column volumes of buffer A (50 mM Hepes, 100 mM NaCl, 10 mM CaCl₂, pH 7.4) then 2 column volumes of buffer B (50 mM Hepes, 100 mM NaCl, 10 mM EDTA, pH 7.4). The method essentially follows the principle described by Thim, L et al. Biochemistry (1988) 27, 7785-779. The products with unfolded Gla-domain was collected and directly applied to a HiTrap Q FF ion-exchange columns (combined CV=5 ml) pre equilibrated in 10 mM His, 100 mM NaCl, pH 7.5. The column was washed with 4 column volumes of 10 mM His, 100 mM NaCl, pH=7.5 and 5 column columns of 10 mM His, 100 mM NaCl, 10 mM CaCl2, pH 7.5 which eluted unmodified FVIIa. The pH was then lowered to 6.0 with 10 mM His, 100 mM NaCl, 10 mM CaCl2, pH 6.0 (4 column volumes). HEPylated FVIIa was eluted with 5 column volumes of 10 mM His, 100 mM NaCl, 10 mM CaCl2, pH 6.0 (60%) and 10 mM His, 1 M NaCl, 10 mM CaCl2, pH 6.0 (40%) buffer mixture. Fractions were combined, and dialyzed against 10 mM His, 100 mM NaCl, 10 mM CaCl2, pH 6.0 using a Slide-A-Lyzer cassette (Thermo Scientific) with a cut-off of 10 kd). Yield (1.4 mg) was determined by quantifying FVIIa against a FVIIa standard using reverse phase HPLC as described above.

### Example 17

### Synthesis of 41.5 kDa HEP-GSC Reagent with 4-Methylbenzoyl Linkage

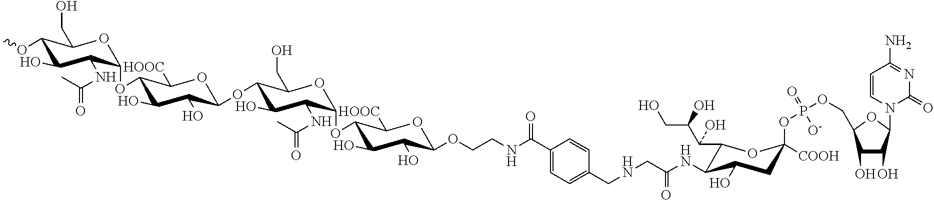

Glycylsialic acid cytidine monophosphate (GSC) (20 mg; 32 μmol) in 5.0 ml 50 mM Hepes, 100 mM NaCl, 10 mM CaCl2 buffer, pH 7.0 was added directly to dry 41.5 kDa HEP-benzaldehyde (99.7 mg; 2.5 l μmol, carbazole quantification assay). The mixture was gently rotated until all HEP-benzaldehyde had dissolved. During the following 2 hours, a 1M solution of sodium cyanoborohydride in MilliQ water was added in portions (5×50 μl), to reach a final concentration of 48 mM. Excess of GSC was then removed by dialysis as follows: the total reaction volume (5250 μl) was transferred to a dialysis cassette (Slide-A-Lyzer Dialysis Cassette, Thermo Scientific Prod#66810 with cut-off 10 kDa capacity: 3-12 ml). Solution was dialysed for 2 hours against 2000 ml of 25 mM Hepes buffer (pH 7.2) and once more for 17 h against 2000 ml of 25 mM Hepes buffer (pH 7.2). Complete removal of excess GSC from inner chamber was verified by HPLC using a Waters X-Bridge phenyl column (4.6 mm×250 mm, 5 μm) and a water acetonitrile system (linear gradient from 0-85% acetonitrile over 30 min containing 0.1% phosphoric acid) using GSC as reference. Inner chamber material was collected and freeze dried to give 83% (carbazole quantification assay) 41.5 kDa HEP-GSC as white powder. The HEP-GSC reagent made by this procedure contains a HEP polymer attached to sialic acid cytidine monophosphate via a 4-methylbenzoyl linkage.

### Example 18

### Synthesis of 21 kDa HEP-GSC Reagent with 4-Methylbenzoyl Linkage

This molecule was prepared using 21 kDa-HEP-benzaldehyde and glycylsialic acid cytidine monophosphate (GSC) in a similar way as described for 41.5 kDa HEP-GSC above. Yield was 78% after freeze drying.

### Example 19

### Desialylation of FVIIa

FVIIa (56.9 mg) was added sialidase (Arthrobacter ureafaciens, 600 μl, 0.3 mg/ml, 200 Um′) in 50 mM Hepes, 150 mM NaCl, 10 mM CaCl₂, pH 7.0 (36 ml), and left for 1 hour at room temperature. The reaction mixture was then diluted with 50 mM Hepes, 150 mM NaCl, pH 7.0 (40 ml), and cooled on ice. 100 mM EDTA solution (6 ml) was added in small portions. After each addition pH was measured. pH should not exceed 9 or fall below 5.5. The EDTA treated sample was then applied to 2×5 ml HiTrap Q FF ion-exchange columns (combined CV=10 ml) pre-equilibrated with 50 mM Hepes, 150 mM NaCl, pH 7.0. Sialidase was eluted with 50 mM Hepes, 150 mM NaCl, pH 7.0 (4 CV), before eluting asialo FVIIa with 50 mM Hepes, 150 mM NaCl, 10 mM CaCl₂, pH 7.0 (10 CV). AsialoFVIIa was isolated in 50 mM Hepes, 150 mM NaCl, 10 mM CaCl₂, pH 7.0. Yield (52.9 mg) and concentration (3.11 mg/ml) was determined by quantifying the FVIIa light chain content against a FVIIa standard after tris(2-carboxyethyl)phosphine reduction using reverse phase HPLC as described above.

### Example 20

### Synthesis of 41.5 kDa-HEP-M-FVIIa with Methylbenzoyl Linkage

To asialo FVIIa (52.9 mg) in 50 mM Hepes, 150 mM NaCl, 10 mM CaCl2, pH 7.0 (17 ml) was added 41.5 kDa-HEP-GSC (90 mg), and rat ST3GalIII enzyme (7 mg; 1.1 unit/mg) in 20 mM Hepes, 120 mM NaCl, 50% glycerol, pH 7.0 (14 ml). 100 mM CaCl2 (4 ml) was then added to raise calcium concentration above 10 mM. The reaction mixture was incubated overnight at 32° C. A solution of 157 mM CMP-NAN in 50 mM Hepes, 150 mM NaCl, 10 mM CaCl2, pH 7.0 (1.1 ml) was added, and the reaction was incubated at 32° C. for an additional hour. HPLC analysis (method described above) showed a product distribution containing un-reacted FVIIa (47%), mono HEPylated FVIIa (40%) and diHEPylated FVIIa (15%) and triHEPylated FVIIa (3%). The reaction mixture was then applied to a FVIIa specific affinity column (CV=72 ml) modified with a Gla-domain specific antibody and step eluted first with 2 column volumes of buffer A (50 mM Hepes, 100 mM NaCl, 10 mM CaCl₂, pH 7.4) then 2 column volumes of buffer B (50 mM Hepes, 100 mM NaCl, 10 mM EDTA, pH 7.4). The method essentially follows the principle described by Thim, L et al. Biochemistry (1988) 27, 7785-779. The products with unfolded Gla-domain was collected and directly applied to 4×5 ml interconnected HiTrap Q FF ion-exchange columns (combined CV=20 ml) equilibrated with a buffer containing 10 mM His, 100 mM NaCl, pH 7.5. The column was washed with 4 column volumes of 10 mM His, 100 mM NaCl, pH 7.5 and 20 column columns of 10 mM His, 100 mM NaCl, 10 mM CaCl₂, pH 7.5 which eluted unmodified FVIIa. The pH was then lowered to 6.0 with 10 mM His, 100 mM NaCl, 10 mM CaCl₂, pH 6.0 (16 column volumes). HEPylated FVIIa was purified by step elution as follows: MonoHEPylated FVIIa was eluted of the column with 20 column volumes of 10 mM His, 100 mM NaCl, 10 mM CaCl2, pH 6.0 (75%) and 10 mM His, 1 M NaCl, 10 mM CaCl2, pH 6.0 (25%) buffer mixture. DiHEPylated FVIIa, containing small amount of monoHEPylated FVIIa was eluted with 20 column volumes of 10 mM His, 100 mM NaCl, 10 mM CaCl2, pH 6.0 (70%) and 10 mM His, 1 M NaCl, 10 mM CaCl2, pH 6.0 (30%) buffer mixture. Fractions containing monoHEPylated FVIIa was combined, and dialyzed against 10 mM His, 100 mM NaCl, 10 mM CaCl2, pH 6.0 using a Slide-A-Lyzer cassette (Thermo Scientific) with a cut-off of 10kD. Yield (7.7 mg) and concentration (0.40 mg/ml) was determined by quantifying the FVIIa light chain content against a FVIIa standard after tris(2-carboxyethyl)phosphine reduction using reverse phase HPLC.

### Example 21

### Synthesis of 21 kDa-HEP-[N]FVIIa with Methylbenzoyl Linkage

To asialo FVIIa (49 mg) in 50 mM Hepes, 150 mM NaCl, 10 mM CaCl2, pH 7.0 (16 ml) was added 21 kDa-HEP-GSC (72 mg), and rat ST3GalIII enzyme (14 mg; 1.1 unit/mg) in 20 mM Hepes, 120 mM NaCl, 50% glycerol, pH 7.0 (20 ml). 100 mM CaCl2 (4 ml) was then added to raise calcium concentration above 10 mM. The reaction mixture was incubated for 18 hours at 32° C. under slow stirring. A solution of 157 mM CMP-NAN in 50 mM Hepes, 150 mM NaCl, 10 mM CaCl2, pH 7.0 (0.2 ml) was then added, and the reaction was incubated at 32° C. for an additional hour. HPLC analysis showed a product distribution containing un-reacted FVIIa (24%), mono HEPylated FVIIa (43%) and diHEPylated FVIIa (25%) and triHEPylated FVIIa (8%). The reaction mixture was applied to a FVIIa specific affinity column (CV=95 ml) modified with a Gla-domain specific antibody and step eluted first with 1½ column volumes of buffer A (50 mM Hepes, 100 mM NaCl, 10 mM CaCl₂, pH 7.4) then 2 column volumes of buffer B (50 mM Hepes, 100 mM NaCl, 10 mM EDTA, pH 7.4). The method essentially follows the principle described by Thim, L et al. Biochemistry (1988) 27, 7785-779. The products with unfolded Gla-domain was collected and directly applied to 4×5 ml connected HiTrap Q FF ion-exchange columns (combined CV=20 ml) equilibrated with a buffer containing 10 mM His, 100 mM NaCl, pH 7.5. The column was washed with 4 column volumes of 10 mM His, 100 mM NaCl, pH 7.5 and 20 column columns of 10 mM His, 100 mM NaCl, 10 mM CaCl2, pH 7.5 which eluted unmodified FVIIa. The pH was then lowered to 6.0 with 10 mM His, 100 mM NaCl, 10 mM CaCl2, pH 6.0 (16 column volumes). Mono-, di- and multiHEPylated FVIIa was separated by step elution using buffer A (10 mM His, 100 mM NaCl, 10 mM CaCl2, pH 6.0) and buffer B (10 mM His, 1 M NaCl, 10 mM CaCl2, pH 6.0). Step elution was as follows: 10 column volumes of 0% B, 20 column volumes of 20% B, 20 colume volumes of 40% B and 40 column volumes of 100% B. Main fractions were analyzed by HPLC, and appropriate mono-, di- and multiHEPylated forms pooled individually. Fractions containing mono-/di- and di-/multi HEPylated FVIIa, was submitted to a second round of anion exchange chromatography as just described, in order to maximize yield of the individual HEPylated forms. Pure isolates were combined, and dialyzed against 10 mM His, 100 mM NaCl, 10 mM CaCl2, pH 6.0 using a Slide-A-Lyzer cassette (Thermo Scientific) with a cut-off of 10kD. In this way 10.97 mg of 21 kDa-HEP-[N]-FVIIa and 4.68 mg of 2×21 kDa-HEP-[N]-FVIIa could be isolated.

### Example 22

### Synthesis of 41.5 kDa HEP-[N]FVIIa L288F T293K with 4-methylbenzoyl Linkage

This material was prepared using FVIIa L288F T293K (32 mg). Protein was initial desialylated as described in example 15, then reacted with 41.5 kDa HEP-GSC (42.0 mg) and ST3GalIII using same procedure as described in example 20. 8.96 mg (28%) 41.5 kDa HEP-[N]-FVIIa L288F T293K was obtained in 10 mM His, 100 mM NaCl, 10 mM CaCl₂, pH 6.0. Unreacted FVIIa L288F T293K mutant was submitted to a second cycle providing an additional 6.34 mg conjugate.

### Example 23

### Synthesis of 41.5 kDa HEP-[N]FVIIa W201 T293K with 4-methylbenzoyl Linkage

This material was prepared by initial desialylation of FVIIa W201R T293K (40 mg) mutant, as described in Example 15. The asialo FVIIa W201R T293K mutant (27.2 mg) thus obtained was reacted with 41.5 kDa HEP-GSC (30.0 mg) and ST3GalIII using same procedure as described in Example 20. 2.9 mg (7.5%) 41.5 kDa HEP-[N]-FVIIa W201 T293K was obtained in 10 mM His, 100 mM NaCl, 10 mM CaCl₂, pH 6.0.

### Example 24

### Synthesis of 41.5 kDa HEP-[N]FVIIa L288F T293K K337A with 4-Methylbenzoyl Linkage

This material was prepared from FVIIa L288F T293K K337A (18.8 mg), by desialylation as described in example 15, followed by reaction with 41.5 kDa HEP-GSC (30.0 mg) and ST3GalIII. The product was purified by affinity chromatography followed by anion exchange chromatography generally as described in example 20. 41.5 kDa HEP-[N]-FVIIa L288F T293K K337A (3.35 mg) was obtained in 10 mM His, 100 mM NaCl, 10 mM CaCl₂, pH 6.0.

### Example 25

### Synthesis of Neuraminic Acid Cytidine Monophosphate Based 41.5 kDa HEP Conjugates with 4-Methylbenzoyl Linkage

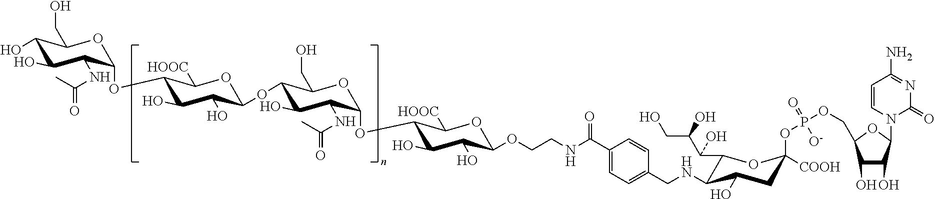

Neuraminic acid cytidine monophosphate is produced as described in Eur. J. Org. Chem. 2000, 1467-1482. Reaction with HEP-aldehyde is performed as described in example 17, replacing GSC with neuraminic acid cytidine monophosphate. Thus, neuraminic acid cytidine monophosphate (32 μmol) is dissolved in 50 mM Hepes, 100 mM NaCl, 10 mM CaCl2 buffer, pH 7.0 buffer and added directly to dry 41.5 kDa HEP-benzaldehyde (2.5 μmol). The mixture is gently rotated until all HEP-benzaldehyde is dissolved. During the following 2 hours, a 1M solution of sodium cyanoborohydride in MilliQ water is added in portions to reach a final concentration of 48 mM. Excess of neuraminic acid cytidine monophosphate is then removed by dialysis as described in example 17. Complete removal of neuraminic acid cytidine monophosphate from inner chamber is verified by HPLC using a Waters X-Bridge phenyl column (4.6 mm×250 mm, 5 μm) and a water acetonitrile system (linear gradient from 0-85% acetonitrile over 30 min containing 0.1% phosphoric acid) using neuraminic acid cytidine monophosphate as reference. Inner chamber material is then collected and freeze dried. The reagent made by this procedure contains a HEP polymer attached to sialic acid cytidine monophosphate via a 4-methylbenzoyl linkage, and is suitable for glycoconjugation to a asialo FVIIa glycoprotein.

### Example 26

### Synthesis of 9-amino-9-deoxy-N-acetylneuraminic acid cytidine monophosphate based HEP conjugates with 4-methylbenzoyl linkage

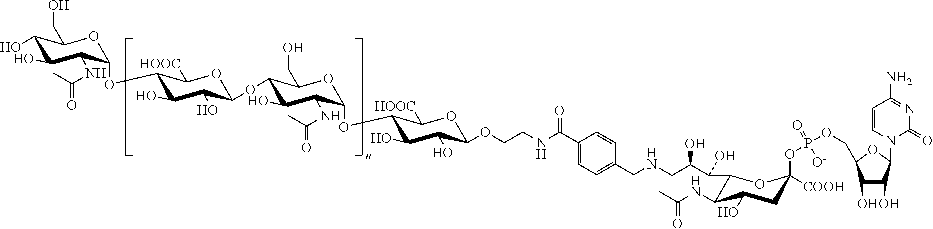

9-deoxy-amino N-acetylneuraminic acid cytidine monophosphate is produced as described in Eur. J. Biochem 168, 594-602 (1987). Reaction with HEP-aldehyde is performed as described in example 17, replacing GSC with 9-amino-9-deoxy-N-acetylneuraminic acid cytidine monophosphate. 9-Amino-9-deoxy-N-acetylneuraminic acid cytidine monophosphate (32 μmol) is dissolved in 50 mM Hepes, 100 mM NaCl, 10 mM CaCl2 buffer, pH 7.0 buffer and added directly to dry 41.5 kDa HEP-benzaldehyde (2.5 μmol). The mixture is gently rotated until all HEP-benzaldehyde is dissolved. During the following 2 hours, a 1M solution of sodium cyanoborohydride in MilliQ water is added in portions to reach a final concentration of 48 mM. Excess of 9-amino-9-deoxy-N-acetylneuraminic acid cytidine monophosphate is then removed by dialysis as described in example 17. Complete removal of 9-amino-9-deoxy-N-acetylneuraminic acid cytidine monophosphate from inner chamber is verified by HPLC on Waters X-Bridge phenyl column (4.6 mm×250 mm, 5 μm) and a water acetonitrile system (linear gradient from 0-85% acetonitrile over 30 min containing 0.1% phosphoric acid) using 9-amino-9-deoxy-N-acetylneuraminic acid cytidine monophosphate as reference. Inner chamber material is collected and freeze dried. The reagent made by this procedure contains a HEP polymer attached to sialic acid cytidine monophosphate via a 4-methylbenzoyl linkage and is suitable for glycoconjugation to a asialo FVIIa glycoprotein.

### Example 27

### Synthesis of 2-keto-3-deoxy-nonic acid cytidine monophosphate based HEP conjugates with 4-methylbenzoyl linkage

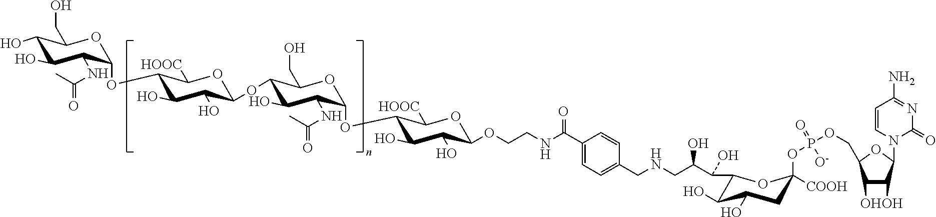

In a way similar to that shown in examples 19 and 20 HEP-sialic acid cytidine monophosphate reagent can be made starting from the sialic acid KDN. The initial amino derivatization at the 9-position is performed as described in Eur. J. Org. Chem. 2000, 1467-1482. Reaction with HEP-aldehyde is performed as described in example 17, replacing GSC with 9-amino-9-deoxy-2-keto-3-deoxy-nonic acid cytidine monophosphate. 9-amino-9-deoxy-2-keto-3-deoxy-nonic acid cytidine monophosphate (32 μmol) is dissolved in 50 mM Hepes, 100 mM NaCl, 10 mM CaCl2 buffer, pH 7.0 buffer and added directly to dry 41.5 kDa HEP-benzaldehyde (2.5 μmol). The mixture is gently rotated until all HEP-benzaldehyde is dissolved. During the following 2 hours, a 1M solution of sodium cyanoborohydride in MilliQ water is added in portions to reach a final concentration of 48 mM. Excess of 9-amino-9-deoxy-2-keto-3-deoxy-nonic acid cytidine monophosphate is then removed by dialysis as described in example 17. Complete removal of 9-amino-9-deoxy-N-acetylneuraminic acid cytidine monophosphate from inner chamber is verified by HPLC on Waters X-Bridge phenyl column (4.6 mm×250 mm, 5 um) and a water acetonitrile system (linear gradient from 0-85% acetonitrile over 30 min containing 0.1% phosphoric acid) using 9-amino-9-deoxy-2-keto-3-deoxy-nonic acid cytidine monophosphate as reference. Inner chamber material is collected and freeze dried. The reagent made by this procedure contains a HEP polymer attached to sialic acid cytidine monophosphate via a 4-methylbenzoyl linkage and is suitable for glycoconjugation to a asialoFVIIa glycoprotein.

### Example 28

### Pharmacokinetic Evaluation in Sprauge Dawley Rats

HEP-FVIIa conjugates were formulated in 10 mM Histidine, 100 mM NaCl, 10 mM CaCl₂, 0.01% Tween80 80, pH 6.0. Sprague Dawley rats (three to six per group) were dosed intravenously with 20 nmol/kg test compound. Stabylite™ (TriniLize Stabylite Tubes; Tcoag Ireland Ltd, Ireland) stabilized plasma samples were collected as full profiles at appropriate time points and frozen until further analysis. Plasma samples were analysed for FVIIa clot activity level using a commercial FVIIa specific clotting assay; STACLOT®IIa-rTF from Diagnostica Stago and antigen concentrations in plasma were determined using LOCI technology. Pharmacokinetic analysis was carried out by non-compartmental methods using Phoenix WinNonlin 6.0 (Pharsight Corporation). Selected parameters are shown in table 2.

TABLE 2
Mean pharmacokinetic parameters of HEP-FVIIa conjugates after IV
administration to Sprague Dawley rats

| Com- | | Cmax | AUC | AUC_{extrapolated} | T½ | MRT |
|---|---|---|---|---|---|---|
| pound | Assay | (nmol/l) | (h * nmol/l) | (%) | (h) | (h) |
| 1 × 40 | LOCI | 337 ± 4 | 4809 ± 58 | 4.3 ± 0.6 | 21.1 ± 0.9 | 25.7 ± 1.1 |
| kDa | | | | | | |
| HEP- | CLOT | 217 ± 10 | 1312 ± 140 | 0.9 ± 0.8 | 5.8 ± 0.6 | 6.5 ± 0.6 |
| [N]- | | | | | | |
| FVIIa | | | | | | |
| 40 kDa | LOCI | 237 ± 18 | 4756 ± 242 | 7.1 ± 1.0 | 26.5 ± 1.8 | 32.8 ± 1.5 |
| PEG- | | | | | | |
| [N]- | CLOT | 222 ± 7 | 1760 ± 61 | 0.9 ± 0.1 | 7.4 ± 0.2 | 8.3 ± 0.3 |
| FVIIa | | | | | | |

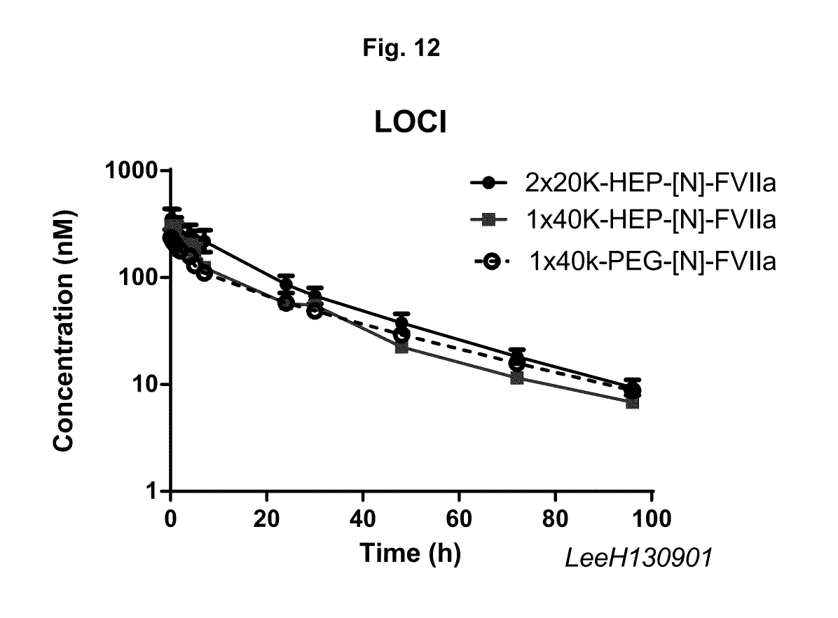

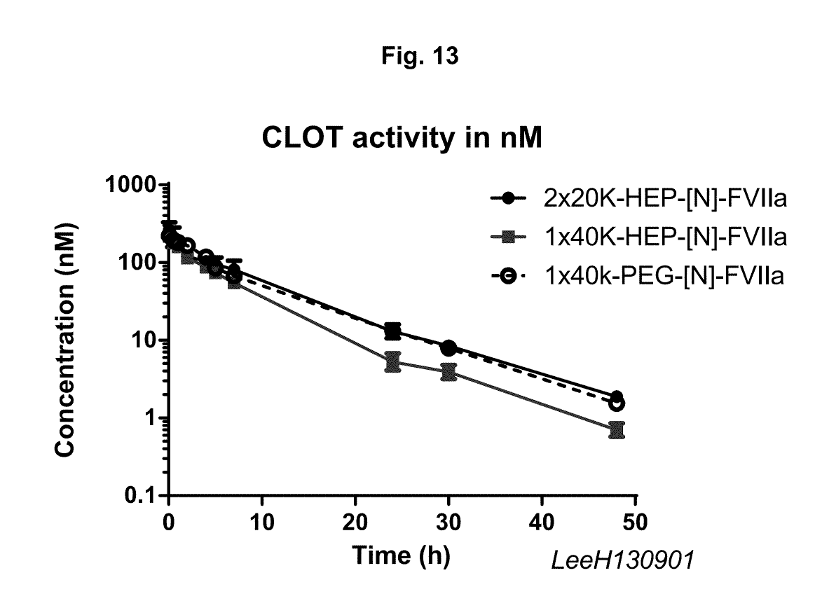

PK-profiles (LOCI and FVIIa:clot) for 40 kDa HEP-[N]-FVIIa and 40 kDa PEG-[N]-FVIIa are shown in FIGS. 12 and 13.

### Example 29

### Plasma Analysis

FVIIa clotting activity levels of 65 kDa HEP-FVIIa 407C conjugates in rat plasma were estimated using a commercial FVIIa specific clotting assay; STACLOT®VIIa-rTF from Diagnostica Stago. The assay is based on the method published by J. H. Morrissey et al, Blood. 81:734-744 (1993). It measures sTF initiated FVIIa activity-dependent time to fibrin clot formation in FVII deficient plasma in the presence of phospholipids. Samples were measured on an ACL9000 coagulation instrument against FVIIa calibration curves with the same matrix as the diluted samples (like versus like). The lower limit of quantification (LLOQ) was estimated to 0.25 U/ml.

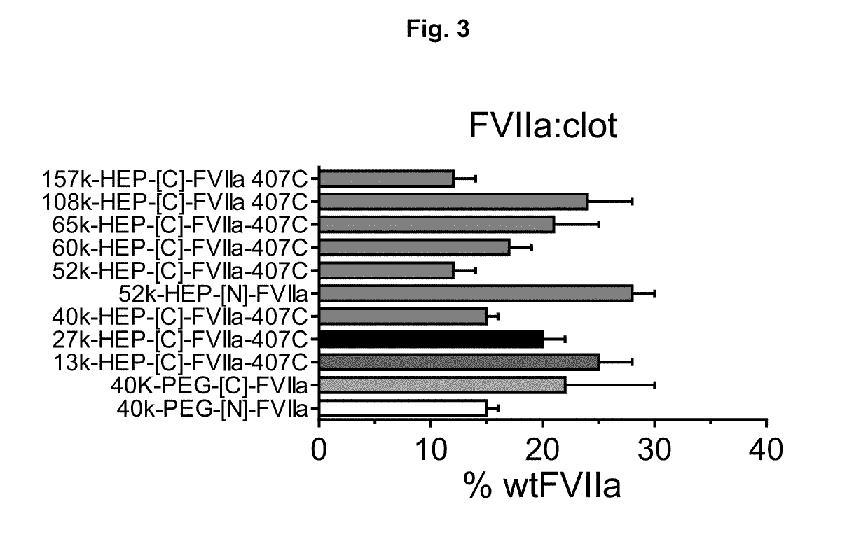

Comparable analysis between cysteine conjugated 13 kDa-, 27 kDa-, 40 kDa-, 52 kDa-, 60 kDa-, 65 kDa-, 108 kDa-, 157 kDa-HEP-[C]-FVIIa407C, glycoconjugated 52 kDa-HEP-[N]-FVIIa and reference molecules (40 kDa-PEG-[N]-FVIIa and 40 kDa-PEG-[C]-FVIIa407C) is shown in FIG. 3. From plasma analysis it is found that heparosan conjugated FVIIa analogues has similar or better activity than the PEG-FVIIa reference molecules.

### Example 30

### Proteolytic Activity Using Plasma-Derived Factor X as Substrate

The proteolytic activity of the HEP-FVIIa conjugates was estimated using plasma-derived factor X (FX) as substrate. All proteins were diluted in 50 mM Hepes (pH 7.4), 100 mM NaCl, 10 mM CaCl₂, 1 mg/mL BSA, and 0.1% (w/v) PEG8000. The kinetic parameters for FX activation were determined by incubating 10 nM of each FVIIa conjugate with 40 nM FX in the presence of 25 μM PC:PS phospholipids (Haematologic technologies) for 30 min at room temperature in a total reaction volume of 100 μL in a 96-well plate (n=2). FX activation in the presence of soluble tissue factor (sTF) was determined by incubating 5 pM of each FVIIa conjugate with 30 nM FX in the presence of 25 μM PC:PS phospholipids for 20 min at room temperature in a total reaction volume of 100 μL (n=2). After incubation, reactions were quenched by adding 50 μL stop buffer [50 mM Hepes (pH 7.4), 100 mM NaCl, 80 mM EDTA] followed by the addition of 50 μL 2 mM chromogenic peptide S-2765 (Chromogenix). Finally, the absorbance increase was measured continuously at 405 nm in a Spectramax 190 microplate reader. Catalytic efficiencies (kcat/Km) were determined by fitting the data to a revised form of the Michaelis Menten equation ([S]<Km) using linear regression. The amount of FXa generated was estimated from a FXa standard curve.

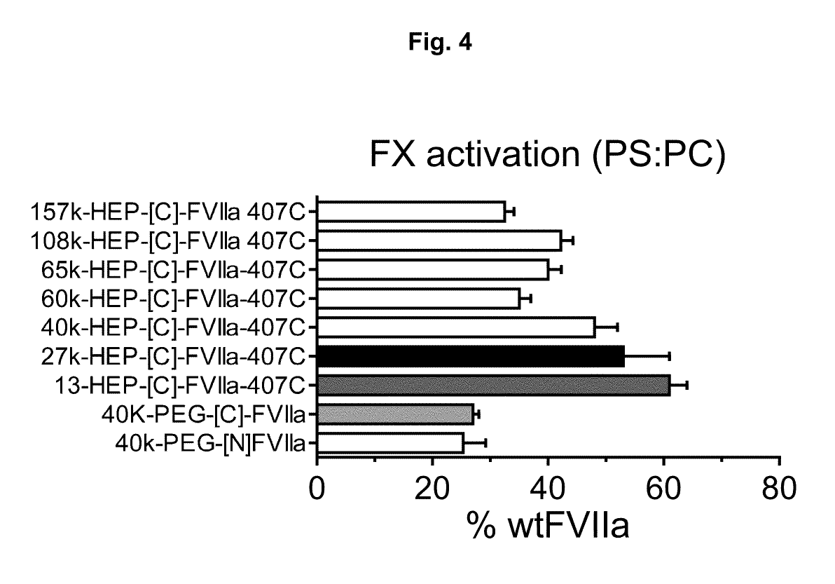

Comparable analysis between 13 kDa, 27 kDa, 40 kDa, 60 kDa, 65 kDa, 108 kDa, 157 kDa-HEP-FVIIa 407C and reference molecules (40 kDa-PEG-[N]-FVIIa and 40 kDa-PEG-[C]-FVIIa407C) is shown in FIG. 4.

Surprisingly, it is found that heparosan cojugated FVIIa analogues all are more active than PEG-FVIIa controls in FX activation assay. For some analogues (e.g. 40 kDa-HEP-FVIIa407C), activity is nearly 2 fold higher than for corresponding 40 kDa-PEG analogues.

### Example 31

### Pharmacokinetic Evaluation in Sprauge Dawley Rats

HEP-FVIIa conjugates were formulated in 10 mM Histidine, 100 mM NaCl, 10 mM CaCl₂, 0.01% Tween80, pH 6.0. Sprague Dawley rats (three to six per group) were dosed intravenously with 20 nmol/kg test compound. Stabylite™ (TriniLize Stabylite Tubes; Tcoag Ireland Ltd, Ireland) stabilized plasma samples were collected as full profiles at appropriate time points and frozen until further analysis. Plasma samples were analysed for FVIIa clot activity level using a commercial FVIIa specific clotting assay; STACLOTNIIa-rTF from Diagnostica Stago and antigen concentrations in plasma were determined using LOCI technology.

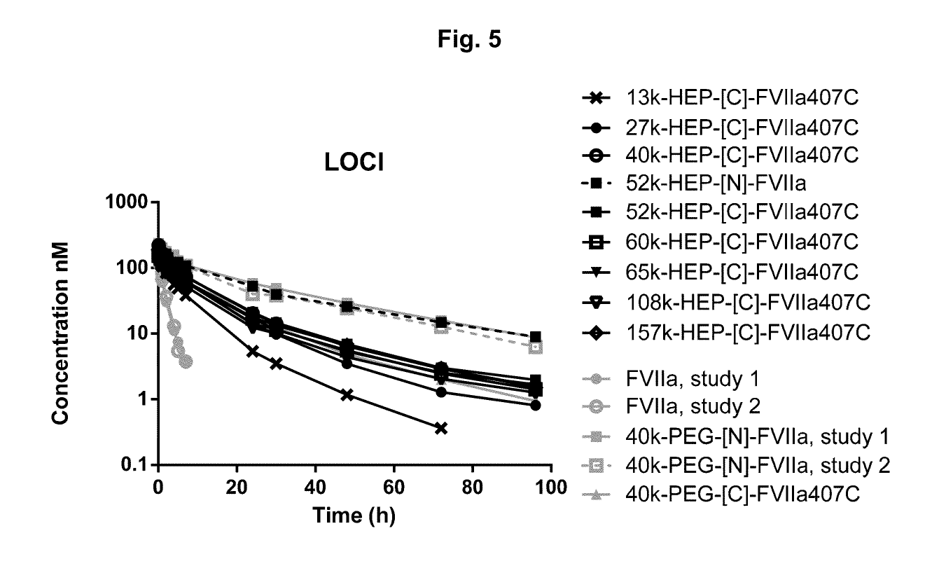

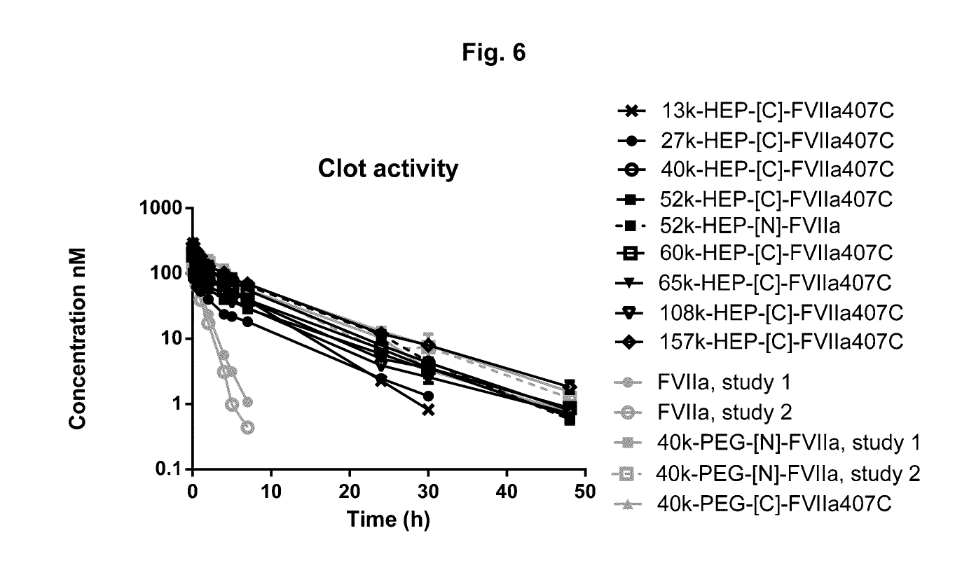

Pharmacokinetic analysis was carried out by non-compartmental methods using Phoenix WinNonlin 6.0 (Pharsight Corporation). The following parameters were estimated: Cmax (maximum concentration) of FVIIa-antithrombin complex, and TV2 (the functional terminal half-life) and MRT (the mean residence time) for clot activity. PK-profiles (LOCI and FVIIa:clot) are shown in FIGS. 5 and 6.

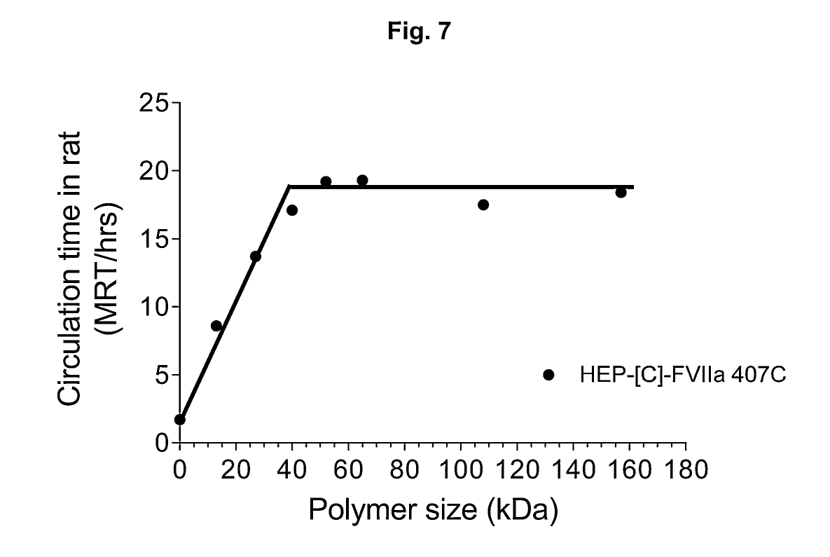

A plot of all LOCI based mean-residence times, as obtained from the non-compartmental analysis methods is shown in FIG. 7.

A linear relation is found between HEP-size and MRT around 13-40 kDa size range. A plateau is reached at approximately 40 kDa HEP-size and beyond.

### EMBODIMENTS

The invention is further described by the following non-limiting embodiments:

In one embodiment the conjugate comprises a FVII polypeptide and a heparosan polymer.

In one embodiment, the heparosan polymer has a mass of between 5 kDa and 200 kDa.

In one embodiment the heparosan polymer has a polydispersity index (Mw/Mn) of less than 1.10.

In one embodiment the heparosan polymer has a polydispersity index (Mw/Mn) of less than 1.07.

In one embodiment the heparosan polymer has a polydispersity index (Mw/Mn) of less than 1.05.

In one embodiment the FVII polypeptide is conjugated to a heparosan polymer having a size of 10 kDa±5 kDa.

In one embodiment the FVII polypeptide is conjugated to a heparosan polymer having a size of 20 kDa ±5 kDa

In one embodiment the FVII polypeptide is conjugated to a heparosan polymer having a size of 30 kDa ±5 kDa.

In one embodiment the FVII polypeptide is conjugated to a heparosan polymer having a size of 40 kDa ±5 kDa.

In one embodiment the FVII polypeptide is conjugated to a heparosan polymer having a size of 50 kDa ±5 kDa.

In one embodiment, the heparosan polymer is branched via a chemical linker. In one embodiment, said heparosan polymers each have a size equal to 20 kDa ±3 kDa.

In one embodiment, said heparosan polymers each have a size equal to 30 kDa ±5 kDa.

In one embodiment, the heparosan polymer is conjugated to FVII polypeptide via an N-glycan.

In one embodiment, one of the two N-glycans at position 145 and 322 are removed by

PNGase F treatment, and heparosan is coupled to the remaining N-glycan.

In another embodiment, the heparosan polymer is conjugated via a sialic acid moiety on FVIIa.

In one embodiment heparosan is coupled to a FVII polypeptide mutant via a single surface exposed cysteine residue.

In one embodiment the heparosan polymer is linked to FVII using a chemical linker comprising 4-methylbenzoyl-GSC.

In one embodiment the heparosan polymer is linked to glycan on the FVII.

In one embodiment a benzaldehyde moiety is attached to the GSC compound, thereby resulting in GSC-benzaldehyde compound suitable for conjugation to a heparosan polymer functionalized with an amine group (cf. FIG. 8).

In one embodiment, 4-formylbenzoic acid is chemically coupled to heparosan and subsequently coupled to GSC by reductive amination (cf. FIG. 9).

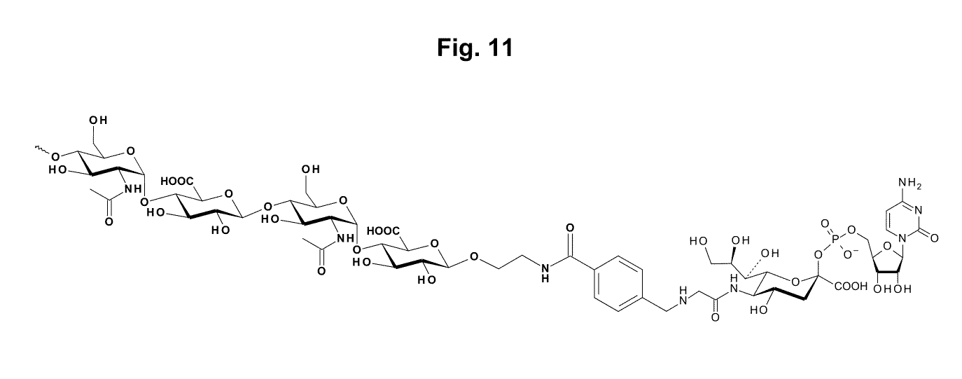

In a preferred embodiment the invention provides GSC-based conjugation wherein a 4-methylbenzoyl moiety is part of the linking structure (cf. FIG. 11).

In one embodiment heparosan comprising a reactive amine is conjugated to a GSC compound functionalized with a benzaldehyde moiety, wherein said amine is reacted with benzaldehyde to yield a (sub)linker between heparosan and GSC which comprises a 4-methylbenzoyl sublinking moiety.

In another embodiment heparosan comprising a reactive benzaldehyde is conjugated to the glycyl amine part of a GSC compound, wherein said benzaldehyde is reacted with an amine to yield a (sub)linker between heparosan and GSC which comprises a 4-methylbenzoyl sublinking moiety.

In one embodiment the conjugate between heparosan and GSC is further conjugated onto FVII to yield a conjugate wherein heparosan is linked to FVII via a 4-methylbenzoyl sublinking moiety and sialic acid derivative.

In one embodiment of the present invention a heparosan polymer is conjugated to a FVII using 4-methylbenzoyl—GSC based conjugation.

In one embodiment, a heparosan polymer moiety comprising an amino group is reacted with 4-formylbenzoic acid and subsequently coupled to the glycyl amino group of GSC by a reductive amination.

In one embodiment GSC prepared by chemoenzymatic route as described in WO07056191 is reacted with a heparosan polymer moiety comprising a benzaldehyde moiety under reducing conditions.

In one embodiment various heparosan-benzaldehyde compounds suitable for coupling to GSC are provided.

In one embodiment the sublinker between heparosan and GSC is not able to form sterio- or regio isomers.

In one embodiment the sublinker between heparosan and GSC is not able to form sterio- or regio isomers, and therefore has lesser potential for generating immune response in humans.

In one embodiment heparosan-GSC is used for preparing a FVII N-glycan HEP conjugate. In one embodiment heparosan-GSC is used for preparing a FVII N-glycan heparosan conjugate using ST3GalIII.

In one embodiment HEP-GSC is used for preparing a FVII 0-glycan HEP conjugate using ST3GalI.

In one embodiment, a CMP activated sialic acid derivative used in the present invention is represented by the following structure:

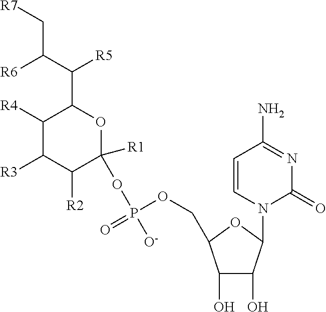

wherein R1 is selected from —COOH, —CONH2, —COOMe, —COOEt, —COOPr and R2, R3, R4, R5, R6 and R7 independently can be selected from —H, —NH2, —SH, —N3, —OH, —F.

In a preferred embodiment, R1 is —COOH, R2 is —H, R3=R5=R6=R7=-OH and R4 is a glycylamido group (—NHC(O)CH2NH2).

In a preferred embodiment the CMP activated sialic acid is GSC having the following structure:

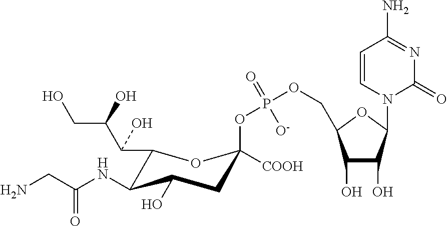

In one embodiment a high yield method for manufacture of HEP having a terminal amine is disclosed.

In one embodiment Factor VII polypeptide is a Factor VII variant comprising two or more substitutions relative to the amino acid sequence of human Factor VII (SEQ ID NO: 1), wherein T293 is replaced by Lys (K), Arg (R), Tyr (Y) or Phe (F); and L288 is replaced by Phe (F), Tyr (Y), Asn (N), Ala (A) or Trp W and/or W201 is replaced by Arg (R), Met (M) or Lys (K) and/or K337 is replaced by Ala (A) or Gly (G).

In some embodiments, the Factor VII polypeptide may comprise a substitution of T293 with Lys (K) and a substitution of L288 with Phe (F). The Factor VII polypeptide may comprise a substitution of T293 with Lys (K) and a substitution of L288 with Tyr (Y). The Factor VII polypeptide may comprise a substitution of T293 with Arg (R) and a substitution of L288 with Phe (F). The Factor VII polypeptide may comprise a substitution of T293 with Arg (R) and a substitution of L288 with Tyr (Y). The Factor VII polypeptide may comprise, or may further comprise, a substitution of K337 with Ala (A). The Factor VII polypeptide may comprise a substitution of T293 with Lys (K) and a substitution of W201 with Arg (R).

The invention is further described by the following non-limiting list of embodiments:
1. A conjugate comprising a Factor VII polypeptide, a linking moiety, and a heparosan polymer wherein the linking moiety between the Factor VII polypeptide and the heparosan polymer comprises X as follows:

[heparosan polymer]-[X]-[Factor VII polypeptide]

wherein X comprises a sialic acid derivative connected to a moiety according to Formula E1 below:

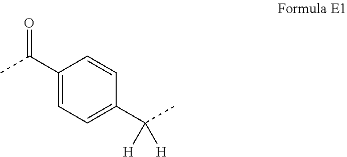

2. The conjugate according to embodiment 1 wherein the sialic acid derivative is a sialic acid derivative according to Formula E2 below:

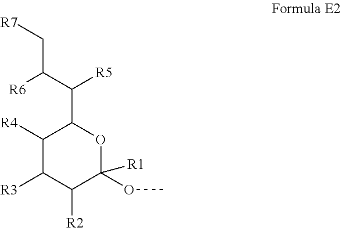

wherein the group in position R1 is selected from the group comprising —COOH, —CONH₂, —COOMe, —COOEt, —COOPr and the group in position R2, R3, R4, R5, R6 and R7 are independently selected from a group comprising —H, —NH—, —NH₂, —SH, —N3, —OH, —F or —NHC(O)CH₂NH—.
3. The conjugate according to embodiment 2 wherein the sialic acid derivative is a glycyl sialic acid according to Formula E3 below:

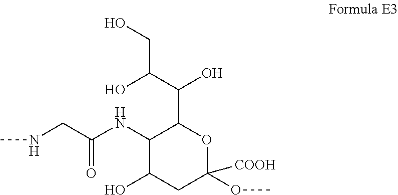

and wherein the moiety of Formula 1 is connected to the terminal —NH handle of Formula E3.
4. The conjugate according to embodiment 1, 2 or 3 wherein

[heparosan polymer]-[X]-

comprises the structural fragment shown in Formula E4 below:

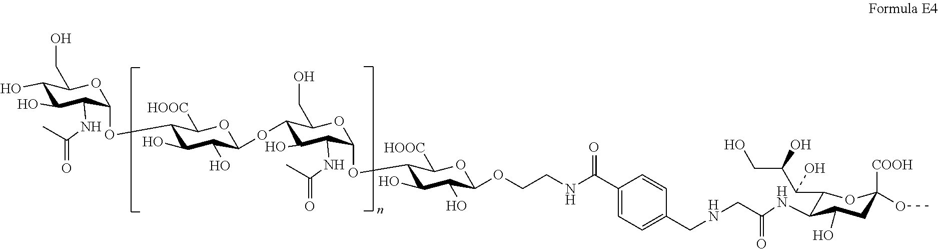

wherein n is an integer from 5 to 450.
5. The conjugate according to any one of embodiments 1 to 4 wherein the heparosan polymer molecular weight is in the range 5 to 100 or 13 to 60 kDa.
6. The conjugate according to embodiment 5 wherein the heparosan polymer molecular weight is in the range 27 to 45 kDa.
7. A pharmaceutical composition comprising the conjugate according to any one of embodiment 1 to 6.
8. Use of a heparosan polymer conjugated to a blood coagulation factor for reducing inter-assay variability in aPTT-based assays.
9. Use according to embodiment 8 wherein the blood coagulation factor is Factor VII.
10. A conjugate according to any one of embodiments 1-6 for use as a medicament.
11. The conjugate according to any one of embodiments 1 to 6 for use in the treatment of coagulopathy.
12. The conjugate according to any one of embodiments 1 to 6 for use in the treatment of haemophilia.
13. The conjugate according to any one of embodiments 1 to 6 for use in prophylactic treatment of haemophilia patients.
14. A conjugate according to any one of embodiments 1 to 6 for use in the treatment of haemophilia wherein the heparosan polymer size is in the range of 5 to 100 kDa.
15. The conjugate according to any one of embodiments 1 to 6 for use in the treatment of haemophilia wherein the heparosan polymer size is in the range of to 60 kDa.
16. The conjugate according to any one of embodiments 1 to 6 for use in the treatment of haemophilia wherein the heparosan polymer size is in the range of 27 to 40 kDa.
17. A method of treating a subject with a coagulopathy comprising administering to said subject the conjugate according to any one of embodiments 1 to 6.
18. A conjugate according to any one of embodiments 1 to 6 for use as a medicament wherein the heparosan polymer molecular weight is in the range of 13 to 60 kDa.
19. Use of a conjugate according to any one of embodiments 1 to 6 for the manufacture of a medicament for use in the treatment of coagulopathy wherein the heparosan polymer molecular weight is in the range of 5 to 100 kDa.
20. Use of a conjugate according to embodiment 19 for the manufacture of a medicament for use in the treatment of coagulopathy wherein the heparosan polymer molecular weight is in the range of 13 to 60 kDa.
21. Use of a conjugate according to embodiment 20 for the manufacture of a medicament for use in the treatment of coagulopathy wherein the heparosan polymer molecular weight is in the range of 27 to 40 kDa.
22. Use according to any one of embodiments 19 to 21 wherein the coagulopathy is haemophilia.
23. Use according to embodiment 22 wherein the coagulopathy is haemophilia A or B.
24. A conjugate comprising a Factor VII polypeptide and a heparosan polymer wherein the heparosan polymer has a molecular weight in the range of 5 to 150 kDa.
25. A conjugate according to embodiment 24 wherein the heparosan polymer weight is 13 to 60 kDa.
26. A conjugate according to embodiment 25 wherein the heparosan polymer weight is 27 to 40 kDa.
27. A conjugate according to embodiment 26 wherein the heparosan polymer weight is 40 to 60 kDa.
28. A method of linking a half-life extending moiety having a reactive amine to a GSC moiety having a reactive amine, wherein the reactive amine on the half-life extending moiety is first reacted with an activated 4-formylbenzoic acid to yield the compound of Formula E5:

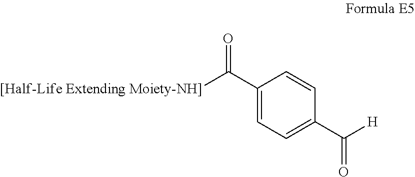

which is subsequently reacted with a GSC moiety under reducing conditions to yield a compound according to Formula E6:

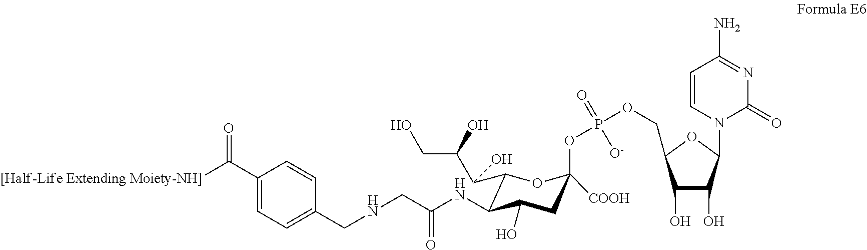

29. A method of linking a half-life extending moiety having a reactive amine to a GSC moiety having a reactive amine, wherein the reactive amine on the GSC moiety first is reacted with an activated 4-formylbenzoic acid to yield a compound according to Formula E7:

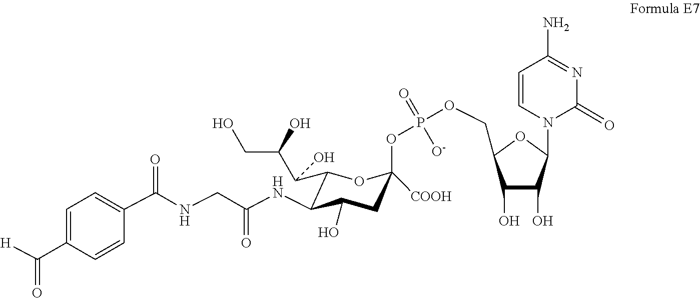

which is subsequently reacted with the reactive amine on the half-life extending moiety under reducing conditions to yield a compound according to Formula E8:

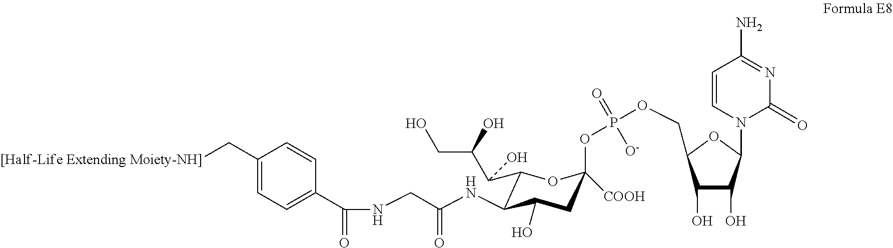

30. The method according to embodiments 28 or 29 wherein the half-life extending moiety is a heparosan polymer.
31. A method according to embodiment 28 wherein a heparosan polymer modified with a 4-formylbenzoyl group (A)

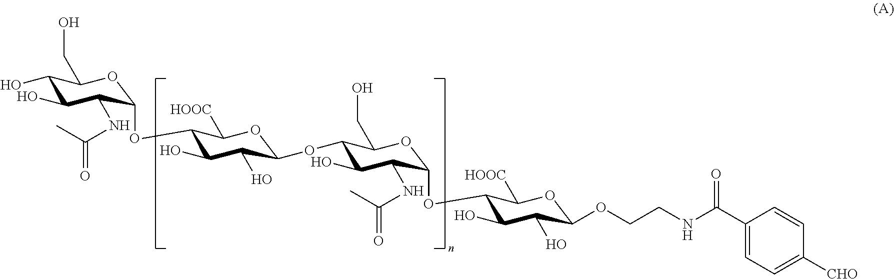

is reacted with GSC (B) in the presence of a reducing agent

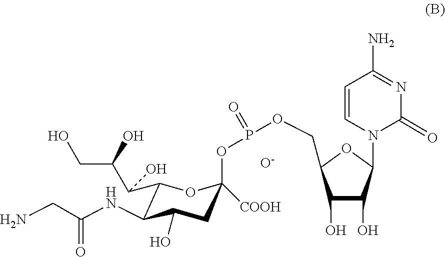

to yield the conjugate (C)

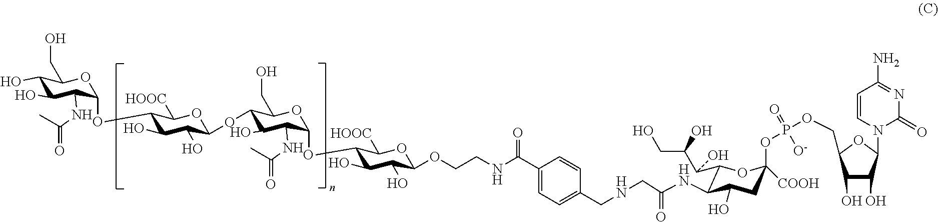

wherein n=5-450.
32. The method according to any one of embodiments 28 to 31 further comprising a subsequent step wherein the half-life extending moiety conjugated to GSC is enzymatically conjugated to Factor VII to yield a conjugate wherein the half-life extending moiety is attached to the protein via a linker comprising a 4-methylbenzoyl sublinker and lacking the cytidine monophosphate group of GSC.
33. A product obtainable by the method according to any one of embodiments 28 to 32.

1. A conjugate comprising a Factor VII polypeptide, a linking moiety, and a heparosan polymer wherein the linking moiety between the Factor VII polypeptide and the heparosan polymer comprises X as follows:
[heparosan polymer]-[X]-[Factor VII polypeptide]
wherein X comprises a sialic acid derivative connected to a moiety according to Formula 1 below:

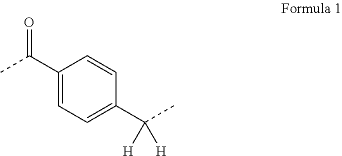

2. The conjugate according to claim 1 wherein the sialic acid derivative is a sialic acid derivative according to Formula 2 below: 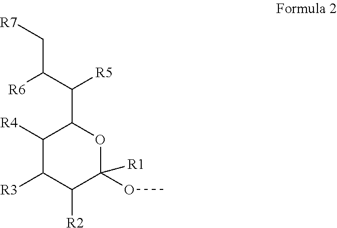
wherein R1 is selected from —COOH, —CONH₂, —COOMe, —COOEt, —COOPr and R2, R3, R4, R5, R6 and R7 are independently selected from —H, —NH₂, —SH, —N3, —OH, and —F.

3. The conjugate according to claim 1 wherein the sialic acid derivative is a glycyl sialic acid according to Formula 3 below: 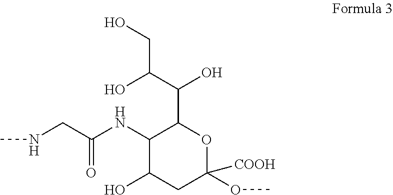
wherein the moiety of Formula 1 is connected to the terminal —NH handle of Formula 3.

4. The conjugate according to claim 1 wherein the
[heparosan polymer]-[X]-
comprises a structure according to Formula 4 below: 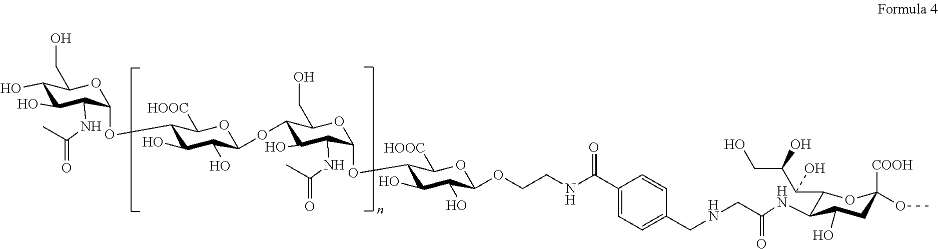
wherein n is an integer from 5 to 450.

5. The conjugate according to claim 1 wherein the heparosan polymer has a molecular weight in the range of 5 to 100 kDa, 13 to 60 kDa, or 27 to 45 kDa.

6. The conjugate according to claim 5 wherein the molecular weight of the heparosan polymer is 40 kDa+/−10%.

7. The conjugate according to claim 1 wherein the Factor VII polypeptide is a Factor VII variant comprising two or more substitutions relative to the amino acid sequence of human Factor VII (SEQ ID NO: 1), wherein T293 is replaced by Lys (K), Arg (R), Tyr (Y) or Phe (F); and L288 is replaced by Phe (F), Tyr (Y), Asn (N), Ala (A) or Trp W and/or W201 is replaced by Arg (R), Met (M) or Lys (K) and/or K337 is replaced by Ala (A) or Gly (G).

8. The conjugate according to claim 1 wherein the Factor VII polypeptide comprise a substitution of T293 with Lys (K) and a substitution of L288 with Phe (F), a substitution of T293 with Lys (K) and a substitution of L288 with Tyr (Y), a substitution of T293 with Arg (R) and a substitution of L288 with Phe (F), a substitution of T293 with Arg (R) and a substitution of L288 with Tyr (Y), or a substitution of T293 with Lys (K) and a substitution of W201 with Arg (R).

9. A pharmaceutical composition comprising the conjugate according to claim 1.

10. A method for reducing inter-assay variability in aPTT-based clotting assays by using a heparosan polymer conjugated to a Factor VII polypeptide.

11. The conjugate according to claim 1 for use as a medicament.

12. A method for treating coagulopathy by using the conjugate according to claim 1.

13. A method for prophylactic or on demand treatment of haemophilia A or B by using the conjugate according to claim 1.

14. A method of conjugating a heparosan polymer to a Factor VII polypeptide comprising the steps of:
a) reacting a heparosan polymer comprising a reactive amine [HEP-NH] with an activated 4-formylbenzoic acid to yield the compound of Formula 5 below, 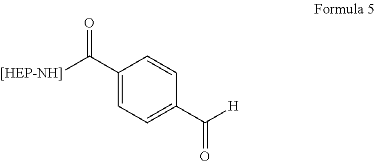
wherein the [HEP-NH is a HEP polymer functionalized with a terminal primary amine,
b) reacting the compound of Formula 5 with a CMP-activated sialic acid derivative under reducing conditions, and
c) conjugating the compound obtained in step b) to a glycan on the Factor VII polypeptide.

15. Conjugates obtainable using the method according to claim 14.


SEQUENCE LISTING (1 sequence)

SEQ ID NO: 1 - protein, 406 aa, Human [sequence omitted]